(19) United States
(12) Patent Application Publication
(10) Pub. No.: US 2008/0070934 A1
(43) Pub. Date: Mar. 20, 2008

(54) KW-3902 CONJUGATES THAT DO NOT CROSS THE BLOOD-BRAIN BARRIER

(76) Inventors: Mark Mugerditichian, Poway, CA (US); Howard Dittrich, San Diego, CA (US); Brian Farmer, San Diego, CA (US); Thomas Murray, Omaha, NE (US); John F.W. Keana, Eugene, OR (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/842,838

(22) Filed: Aug. 21, 2007

Related U.S. Application Data
(60) Provisional application No. 60/839,457, filed on Aug. 22, 2006.
(60) Provisional application No. 60/942,415, filed on Jun. 6, 2007.

(51) Int. Cl. A61K 31/522; A61P 13/12; A61P 7/10; C07D 473/04

(52) U.S. Cl. 514/263.34; 544/267

(57) ABSTRACT

The present invention relates to certain compounds and to methods for the preparation of certain compounds that can be used in the fields of chemistry and medicine. Specifically, described herein are methods for the preparation of various compounds and intermediates, and the compounds and intermediates themselves. More specifically, described herein are methods for synthesizing KW-3902 derivatives of Formula (I), (II), (III), (IV), (V), and (VI).

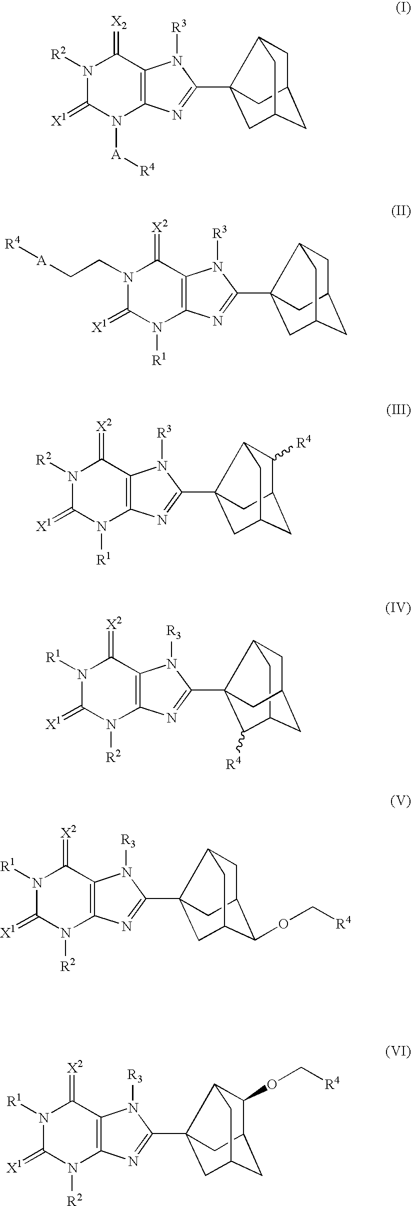

### RELATED APPLICATIONS

The present application claims priority to U.S. Provisional Application Ser. No. 60/839,457, filed on Aug. 22, 2006, by Dittrich et al., and entitled “KW-3902 CONJUGATES THAT DO NOT CROSS THE BLOOD BRAIN BARRIER,” and to U.S. Provisional Application Ser. No. 60/942415, filed on Jun. 6, 2007, by Mugerditchian et al., and entitled “KW-3902 CONJUGATES THAT DO NOT CROSS THE BLOOD BRAIN BARRIER,” the entire disclosures of which are herein incorporated by reference in their entireties.

### BACKGROUND OF THE INVENTION

1. Field of the Invention

The present invention relates to certain compounds and to methods for the preparation of certain compounds that can be used in the field of chemistry and medicine.

2. Description of the Related Art

Adenosine is involved in the regulation of renal haemodynamics, tubular reabsorption of fluid and solutes, and in renin release in kidneys. In contrast to other vascular beds, adenosine induces vasoconstriction in the kidney, thereby coupling renal perfusion to the metabolic rate of the organ. In addition to its renal and diuretic effects, adenosine modulates seizures. Seizures and convulsions are the consequence of temporary abnormal electrophysiologic phenomena of the brain, resulting in abnormal synchronization of electrical neuronal activity. Every individual has a seizure threshold, i.e., a tolerance point beyond which a seizure can be induced. For example, individuals who develop seizure disorders have a lower threshold for seizures than others. Sleep deprivation, prolonged or acute stress, exhaustion, fear, illness, increases in breathing rates or changes in blood sugar levels are exemplary factors known to lower the seizure threshold.

Adenosine exerts its biologic functions through binding to different G-Protein Coupled Receptors (“GPCRs”), e.g., A₁, A_{2A}, A_{2B}, A₃ and A₄. The adenosine A₁ receptor regulates renal fluid balance, as well as excitatory glutamatergic neurotransmission, which contributes to its anticonvulsant activity. Antagonists to A₁ receptors (AA₁RAs) cause diuresis and natriuresis without major changes in glomerular filtration rate (“GFR”) and decrease afferent arteriolar pressure. Xanthine-derived adenosine A₁ receptor antagonists, such as KW-3902, are effective diuretics, renal-protectants, and bronchiodialors, also lower the seizure threshold of individuals.

The chemical name of the AA₁RA KW-3902 is 8-(3-noradamantyl)-1,3-dipropylxanthine, also known as 3,7-dihydro-1,3-dipropyl-8-(3-tricyclo[3.3.1.0^{3,7}]nonyl)-1H-purine-2,6-dione, and its structure is

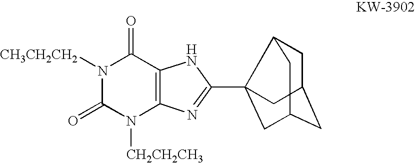

KW-3902 and related compounds are described, for example, in U.S. Pat. Nos. 5,290,782, 5,395,836, 5,446,046, 5,631,260, 5,736,528, 6,210,687, and 6,254,889, the entire disclosure of all of which are hereby incorporated by reference herein, including any drawings.

KW-3902 and related compounds have a diuretic effect, a renal-protecting effect, and a bronchiodilatory effect. Further, KW-3902, when combined with a standard diuretic is beneficial to subjects who are refractory to standard therapy. KW-3902 also blocks the tubuloglomerular feedback (“TGF”) mechanism mediated by adenosine (via A₁ receptors) described above. This ultimately allows for increased GFR and improved renal function, which results in more fluid passing through the loop of Henle and the distal tubule. In addition, KW-3902 inhibits the reabsorption of sodium (and, therefore, water) in the proximal tubule, which results in diuresis. Furthermore, KW-3902 is an inhibitor of TGF, which can counteract the adverse effect of some diuretics, such as proximal diuretics, which activate or promote TGF. See, e.g., U.S. Pat. No. 5,290,782, and U.S. patent application Ser. No. 10/830,617 filed Apr. 23, 2004, Ser. No. 11/248,479 filed Oct. 11, 2005, Ser. No. 11/248,905 filed Oct. 11, 2005, and Ser. No. 11/464,665, filed Jun. 16, 2006 the entire disclosure of all of which are hereby incorporated by reference herein, including any drawings.

There is a need for compounds that retain the ability to function as effective adenosine A₁ receptor antagonists but that have a reduced ability to cross the blood brain barrier. Such compounds can retain the diuretic and renal-protective functions of AA₁RAs, but would reduce or eliminate the undesirable central nervous system (CNS) side effects of adenosine A₁ receptor antagonism.

### SUMMARY OF THE INVENTION

Embodiments disclosed herein generally relate to the synthesis of chemical compounds, including xanthine derivatives that do not substantially cross the blood-brain barrier. Some embodiments are directed to the chemical compound and intermediate compounds. Other embodiments are directed to the individual methods of synthesizing chemical compounds and intermediates disclosed herein. Still other embodiments relate to methods of using the chemical compounds described herein.

Embodiments disclosed herein relate to the compounds of Formula (I), (II), (III), (IV), (V) and (VI), or pharmaceutically acceptable salts, esters, metabolites, or prodrugs thereof.

Other embodiments disclosed herein relate to the individual methods of synthesizing compounds of Formula (I), (II), (III), (IV), (V) and (VI).

Yet other embodiments relate to pharmaceutical compositions comprising a compound of Formula (I), (II), (III), (IV), (V) or (VI), or pharmaceutically acceptable salts, esters, metabolites, or prodrugs thereof, and a non adenosine-modifying diuretic.

Other embodiments relate to methods of inducing diuresis in a subject in need thereof, by identifying a subject in need thereof, and providing to the subject a therapeutically effective amount of a compound of Formula (I), (II), (III), (IV), (V) or (VI) or a pharmaceutically acceptable salt, ester, metabolite, or prodrug thereof and a non adenosine-modifying diuretic.

Yet other embodiments relate to methods of maintaining or restoring the diuretic effect of a non-adenosine modifying diuretic in a subject by providing a compound of Formula (I), (II), (III), (IV), (V) or (VI), or a pharmaceutically acceptable salt, ester, metabolite, or prodrug thereof and a non adenosine-modifying diuretic.

Still other embodiments relate to methods of improving renal function by identifying a subject suffering from impaired creatinine clearance and providing to said subject an amount of Formula (I), (II), (III), (IV), (V) or (VI), or a pharmaceutically acceptable salt, ester, metabolite, or prodrug thereof effective to maintain or increase creatinine clearance, and a non adeno sine-modifying diuretic.

Other embodiments relate to methods of maintaining renal function by identifying a subject with impaired creatinine clearance and providing to the subject an amount of Formula (I), (II), (III), (IV), (V) or (VI), or a pharmaceutically acceptable salt, ester, metabolite, or prodrug thereof and a non adenosine-modifying diuretic, thereby slowing or arresting the impairment in creatinine clearance for a period of time.

Other embodiments relate to methods of restoring renal function, by identifying a subject having increased serum creatinine levels and/or decreased creatinine clearance and providing to said subject an amount of a compound of Formula (I), (II), (III), (IV), (V) or (VI), or a pharmaceutically acceptable salt, ester, metabolite, or prodrug thereof, and a non adenosine-modifying diuretic, thereby decreasing serum creatinine levels and/or slowing or arresting the impairment of creatinine clearance.

Yet other embodiments relate to methods of improving, maintaining, or restoring renal function by identifying a subject suffering from congestive heart failure and renal impairment and providing to the subject an amount of a compound of Formula (I), (II), (II), (IV), (V) or (VI), or a pharmaceutically acceptable salt, ester, metabolite, or prodrug thereof, and a non adenosine-modifying diuretic.

Still other embodiments relate to methods of improving, maintaining, or restoring renal function in a subject by identifying a subject that is suffering from congestive heart failure who is refractory to standard diuretic therapy and providing to the subject an amount of a compound of Formula (I), (II), (III), (IV), (V) or (VI), or a pharmaceutically acceptable salt, ester, metabolite, or prodrug thereof, effective to maintain or increase creatinine clearance, and a diuretic.

Yet other embodiments relate to methods of treating acute fluid overload in a subject by identifying a subject in need of short-term hospitalization to treat acute fluid overload, hospitalizing said subject, providing the subject with a non adenosine-modifying diuretic and an amount compound of Formula (I), (II), (III), (IV), (V), or (VI) or a pharmaceutically acceptable salt, ester, metabolite, or prodrug thereof effective to accelerate removal of excess fluid from the subject compared to diuretic therapy alone.

Still other embodiments relate to methods of improving, maintaining, or restoring renal function in subjects with stable congestive heart failure taking chronic diuretics by identifying a subject with stable congestive heart failure taking chronic diuretics and providing to the subject a therapeutically effective amount of a compound of Formula (I), (II), (III), (IV), (V) or (VI), or a pharmaceutically acceptable salt, ester, metabolite, or prodrug thereof in about four day to about monthly intervals, wherein the subject simultaneously continues the chronic diuretic therapy throughout the course of treatment with the compound of Formula (I), (II), (III), (IV), (V), or (VI).

### DETAILED DESCRIPTION OF THE PREFERRED EMBODIMENT

KW-3902 and its analogs have numerous biological activities, described for example in U.S. Pat. Nos. 5,290,782, 5,395,836, 5,446,046, 5,631,260, 5,736,528, 6,210,687, 6,254,889, and co-pending U.S. patent application Ser. No. 10/830,617 filed Apr. 23, 2004, Ser. No. 11/248,479 filed Oct. 11, 2005, Ser. No. 11/248,905 filed Oct. 11, 2005, and Ser. No. 11/464,665, filed Jun. 16, 2006 the entire disclosure of all of which are hereby incorporated by reference herein, including any drawings.

Provided herein are derivatives of KW-3902, as well as pharmaceutically acceptable salts, esters, amides, metabolites and prodrugs thereof that have substituted xanthine or noradamantyl rings, methods of their synthesis, and methods of their use. The derivatives of KW-3902 can have a polar or charged moiety linked to either the xanthine ring or the noradamantyl group of KW-3902.

Metabolites of KW-3902 are known. These include compounds where the propyl groups on the xanthine entity are hydroxylated, or that the propyl group is an acetylmethyl (CH₃C(O)CH₂—) group. Other metabolites include those in which the noradamantyl group is hydroxylated (i.e., is substituted with a —OH group) or oxylated (i.e., is substituted with a ═O group). Thus, examples of metabolites of KW-3902 include, but are not limited to, 8-(trans-9-hydroxy-3-tricyclo[3.3.1.0^{3,7}]nonyl)-1,3-dipropylxanthine (also referred to herein as “M1-trans”), 8-(cis-9-hydroxy-3-tricyclo[3.3.1.0^{3,7}]nonyl)-1,3-dipropylxanthine (also referred to herein as “M1-cis”), 8-(trans-9-hydroxy-3-tricyclo[3.3.1.0^{3,7}]nonyl)-1-(2-oxopropyl)-3-propylxanthine and 1-(2-hydroxypropyl)-8-(trans-9-hydroxy-3-tricyclo[3.3.1.0^{3,7}]nonyl)-3 -propylxanthine.

Any amine, hydroxy, or carboxyl side chain on the metabolites, esters, or amides of the above compounds can be esterified or amidified. The procedures and specific groups to be used to achieve this end is known to those of skill in the art and can readily be found in reference sources such as Greene and Wuts, Protective Groups in Organic Synthesis, 3^{rd} Ed., John Wiley & Sons, New York, N.Y., 1999, which is incorporated herein in its entirety. Thus some embodiments relate to derivatives of KW-3902 metabolites.

A “prodrug” refers to an agent that is converted into the parent drug in vivo. Prodrugs are often useful because, in some situations, they may be easier to administer than the parent drug. They may, for instance, be bioavailable by oral administration whereas the parent is not. The prodrug may also have improved solubility in pharmaceutical compositions over the parent drug. An example, without limitation, of a prodrug would be a compound of the present invention which is administered as an ester (the “prodrug”) to facilitate transmittal across a cell membrane where water solubility is detrimental to mobility but which then is metabolically hydrolyzed to the carboxylic acid, the active entity, once inside the cell where water-solubility is beneficial. A further example of a prodrug might be a short peptide (polyaminoacid) bonded to an acid group where the peptide is metabolized to reveal the active moiety. Thus, some embodiments relate to derivatives of KW-3902 prodrugs.

As stated above the KW-3902 derivatives disclosed herein can have a charged or polar moiety linked to the xanthine ring and/or adamantyl group. Polar moieties are characxterized by the presence of δ+ and δ− charges within the chemical group owing to electronegativity differences between pair(s) of atoms bonded together. Charged moieties refer to any chemical moiety that is either partially or completely ionized under physiological conditions, typically owing to either protonation in the case of a basic moiety or loss of one or more proteins in the case of an acidic moiety. Exemplary polar and charged moieties useful in the embodiments described herein include but are not limited to —SO₃⁻ (sulfonate), —OSO₃⁻ (sulfate), —SO₂⁻ (sulfinate), —CO₂⁻ (carboxylate), PO₃²⁻ (phosphonate), —OPO₃²⁻ (phosphate monoester), —OP(O₂)OR⁻ (phosphate diester), —PO₂⁻ (phosphinate), —B(OH)O⁻ (borate), —O(CH₂CH₂0)ₙH (a PEG), —ONO₂ (nitrate ester), —ONO (nitrite ester), —CF₃, —CH₂F, —CHF₂, cyano, isocyano, amide, guanadinium, and amino groups.

Various types of linkers can exist between the polar or charged moiety and the xanthine or noradamantyl group. For example, the groups can be linked via a substituted or unsubstituted branched or unbranched alkyl, alkenyl, alkynyl, ester, ether, thioether, amide, or other type of linkage. In some embodiments, the linker can comprise a combination of carbon chain moieties (e.g., —CH₂— or CH═, or carbonyl) with ester, ether, thioether, or amide linkages. In preferred embodiments, the linker is between 0-10 residues, for example 1, 2, 3, 4, 5, 6, 7, 8, 9, 10 or more up to 12, 14, 16, 18, or 20 (or even more) residues. Some embodiments relate to KW-3902 derivatives with a polar or charged moiety linked to the xanthine ring and/or the noradamantyl of KW-3902 via a C—C linkage. Some embodiments relate to KW-3902 derivatives with a polar or charged moiety linked to the noradamantyl group via an ether bridge.

### KW-3902 Derivatives with Substitutions on the Xanthine Ring

Accordingly, some embodiments provide derivatives of KW-3902 of Formula (I) or (II), and methods of their synthesis:

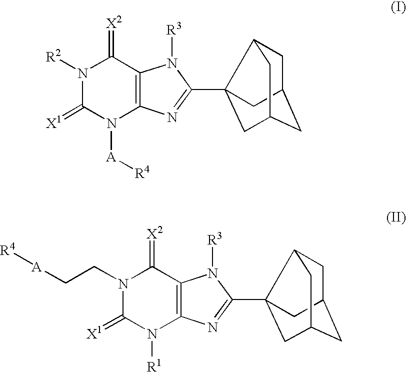

wherein R¹ and R² represent a hydrogen, lower alkyl, allyl, propargyl, or hydroxyl-substituted, oxo-substituted, or unsubstituted lower alkyl, and R³ represents hydrogen or lower alkyl, wherein each of X¹ and X² independently represents oxygen or sulfur, wherein A is a substituted or unsubstituted substituent selected from the group consisting of a branched or unbranched alkyl, an alkenyl, an alkynyl, an ester, an ether, a thioether, an amide, or an arylamide. R⁴ represents an aryl, heteroaryl, heterocyclyl, cycloalkyl, cycloalkenyl, cycloalkynyl, arylalkyl, heteroarylalkyl, and heterocyclylalkyl, wherein each of the foregoing is substituted with a charged or polar moiety, or a pharmaceutically acceptable salt, ester, amide, metabolite, or prodrug thereof

### KW-3902 Derivatives with C—C Substitutions on the Noradamantyl Ring

Some embodiments provide derivatives of KW-3902 of Formula (III) or (IV), and methods of their synthesis:

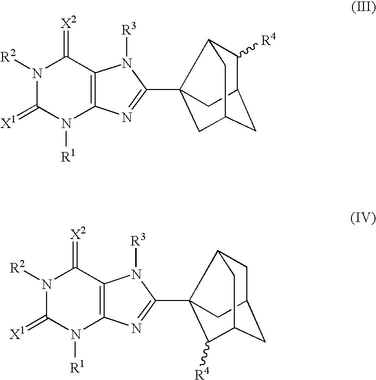

wherein each of R¹, R² independently represents hydrogen, lower alkyl, allyl, propargyl, or hydroxy-substituted, oxo-substituted or unsubstituted lower alkyl, and R³ represents hydrogen or lower alkyl, and wherein each of X¹ and X² independently represents oxygen or sulfur. R⁴ represents an aryl, heteroaryl, heterocyclyl, cycloalkyl, cycloalkenyl, cycloalkynyl, arylalkyl, heteroarylalkyl, and heterocyclylalkyl, wherein each of the foregoing is substituted with a charged or polar moiety, or a pharmaceutically acceptable salt, ester, amide, metabolite, or prodrug thereof.

### KW-3902 Derivatives with Ether-Linked Substitutions on the Noradamantyl Ring

As discussed further below, other embodiments provide derivatives of KW-3902 of Formula (V) or (VI), having a substitution on the noradamantyl ring via an ether linkage and method of their synthesis:

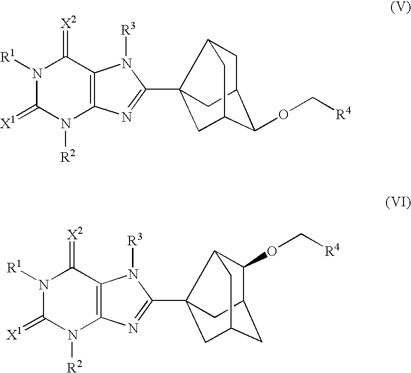

wherein each of R¹, R² independently represents hydrogen, lower alkyl, allyl, propargyl, or hydroxy-substituted, oxo-substituted or unsubstituted lower alkyl, and R₃ represents hydrogen or lower alkyl, and wherein each of X¹ and ² independently represents oxygen or sulfur. R⁴ represents an aryl, heteroaryl, heterocyclyl, cycloalkyl, cycloalkenyl, cycloalkynyl, arylalkyl, heteroarylalkyl, and heterocyclylalkyl, wherein each of the foregoing is substituted with a charged or polar moiety, or a pharmaceutically acceptable salt, ester, arnide, metabolite, or prodrug thereof.

For the compounds described herein, each stereogenic carbon can be of R or S configuration. Although the specific compounds exemplified in this application can be depicted in a particular configuration, compounds having either the opposite stereochemistry at any given chiral center or mixtures thereof are also envisioned unless otherwise specified. When chiral centers are found in the derivatives of this invention, it is to be understood that the compounds encompasses all possible stereoisomers unless otherwise indicated.

As used herein, any “R” group(s) such as, without limitation, R, R¹, R², R³, R⁴, represent substituents that can be attached to the indicated atom. An R group may be substituted or unsubstituted. If two “R” groups are covalently bonded to the same atom or to adjacent atoms, then they may be “taken together” as defined herein to form a cycloalkyl, aryl, heteroaryl or heterocycle. For example, without limitation, if R₁ₐ and R_{1b} of an NR₁ₐR_{1b} group are indicated to be “taken together,” it means that they are covalently bonded to one another at their terminal atoms to form a ring:

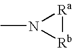

The term “alkyl,” as used herein, means any unbranched or branched, substituted or unsubstituted, saturated hydrocarbon, with C₁-C₂₄ preferred, and C₁-C₆ hydrocarbons being preferred, with methyl, ethyl, propyl, isopropyl, butyl, isobutyl, and tert-butyl, and pentyl being most preferred.

The term “alkenyl,” and all E,Z stereoisomers thereof as used herein, means any unbranched or branched, substituted or unsubstituted, unsaturated hydrocarbon containing one or more double bonds. Some examples of alkenyl groups include allyl, homo-allyl, vinyl, crotyl, butenyl, pentenyl, hexenyl, heptenyl and octenyl.

The term “alkynyl” as used herein, means any unbranched or branched, substituted or unsubstituted, unsaturated hydrocarbon with one or more triple bonds.

The term “cycloalkyl” refers to any non-aromatic, substituted or unsubstituted, hydrocarbon ring, preferably having five to twelve atoms comprising the ring. Furthermore, in the present context, the term “cycloalkyl” comprises fused ring systems such that the definition covers bicyclic and tricyclic structures.

The term “cycloalkenyl” refers to any non-aromatic, substituted or unsubstituted, hydrocarbon ring that includes a double bond, preferably having five to twelve atoms comprising the ring. Furthermore, in the present context, the term “cycloalkenyl” comprises fused ring systems such that the definition covers bicyclic and tricyclic structures.

The term “cycloalkynyl” refers to any non-aromatic, substituted or unsubstituted, hydrocarbon ring that includes a triple bond, preferably having five to twelve atoms comprising the ring. Furthermore, in the present context, the term “cycloalkynyl” comprises fused ring systems such that the definition covers bicyclic and tricyclic structures.

The term “acyl” refers to hydrogen, lower alkyl, lower alkenyl, or aryl connected, as substituents, via a carbonyl group. Examples include formyl, acetyl, propanoyl, benzoyl, and acryl. An acyl may be substituted or unsubstituted.

In the present context the term “aryl” is intended to mean a carbocyclic aromatic ring or ring system. Moreover, the term “aryl” includes fused ring systems wherein at least two aryl rings, or at least one aryl and at least one C₃₋₈-cycloalkyl share at least one chemical bond. Some examples of “aryl” rings include optionally substituted phenyl, naphthalenyl, phenanthrenyl, anthracenyl, tetralinyl, fluorenyl, indenyl, and indanyl. An aryl group may be substituted or unsubstituted.

In the present context, the term “heteroaryl” is intended to mean a heterocyclic aromatic group where one or more carbon atoms in an aromatic ring have been replaced with one or more heteroatoms selected from the group comprising nitrogen, sulfur, phosphorous, and oxygen. Furthermore, in the present context, the term “heteroaryl” comprises fused ring systems wherein at least one aryl ring and at least one heteroaryl ring, at least two heteroaryl rings, at least one heteroaryl ring and at least one heterocyclyl ring, or at least one heteroaryl ring and at least one C₃₋₈-cycloalkyl ring share at least one chemical bond. A heteroaryl can be substituted or unsubstituted.

The terms “heterocycle” and “heterocyclyl” are intended to mean three-, four-, five-, six-, seven-, and eight-membered rings wherein carbon atoms together with from 1 to 3 heteroatoms constitute said ring. A heterocycle may optionally contain one or more unsaturated bonds situated in such a way, however, that an aromatic π-electron system does not arise. The heteroatoms are independently selected from oxygen, sulfur, and nitrogen. A heterocycle may further contain one or more carbonyl or thiocarbonyl functionalities, so as to make the definition include oxo-systems and thio-systems such as lactams, lactones, cyclic imides, cyclic thioimides, cyclic carbamates, and the like. Heterocyclyl rings may optionally also be fused to at least other heterocyclyl ring, at least one C₃₋₈-cycloalkyl ring, at least one C₃₋₈-cycloalkenyl ring and/or at least one C₃₋₈-cycloalkynyl ring such that the definition includes bicyclic and tricyclic structures. Examples of benzo-fused heterocyclyl groups include, but are not limited to, benzimidazolidinone, tetrahydroquinoline, and methylenedioxybenzene ring structures. Some examples of “heterocycles” include, but are not limited to, tetrahydrothiopyran, 4H-pyran, tetrahydropyran, piperidine, 1,3-dioxin, 1,3-dioxane, 1,4-dioxin, 1,4-dioxane, piperazine, 1,3-oxathiane, 1,4-oxathiin, 1,4-oxathiane, tetrahydro-1,4-thiazine, 2H-1,2-oxazine, maleimide, succinimide, barbituric acid, thiobarbituric acid, dioxopiperazine, hydantoin, dihydrouracil, morpholine, trioxane, hexahydro-1,3,5-triazine, tetrahydrothiophene, tetrahydrofuran, pyridine, pyridinium, pyrroline, pyrrolidine, pyrrolidone, pyrrolidione, pyrazoline, pyrazolidine, imidazoline, imidazolidine, 1,3-dioxole, 1,3-dioxolane, 1,3-dithiole, 1,3-dithiolane, isoxazoline, isoxazolidine, oxazoline, oxazolidine, oxazolidinone, thiazoline, thiazolidine, and 1,3-oxathiolane. A heterocycle group of this invention may be substituted or unsubstituted.

The term “alkoxy” refers to any unbranched, or branched, substituted or unsubstituted, saturated or unsaturated ether, with C₁-C₆ unbranched, saturated, unsubstituted ethers being preferred, with methoxy being preferred.

The term “cycloalkoxy” refers to any non-aromatic hydrocarbon ring that is attached to an oxygen atom, which is itself attached to another carbon atom in the molecule. A cycloalkoxy can be substituted or unsubstituted.

The term “alkoxy carbonyl” refers to any linear, branched, cyclic, saturated, unsaturated, aliphatic or aromatic alkoxy or hetroalkoxy attached to a carbonyl group. The examples include methoxycarbonyl group, ethoxycarbonyl group, propyloxycarbonyl group, isopropyloxycarbonyl group, butoxycarbonyl group, sec-butoxycarbonyl group, tert-butoxycarbonyl group, cyclopentyloxycarbonyl group, cyclohexyloxycarbonyl group, benzyloxycarbonyl group, allyloxycarbonyl group, phenyloxycarbonyl group, pyridyloxycarbonyl group, and the like. An alkoxy carbonyl may be substituted or unsubstituted.

The term “(cycloalkyl)alkyl” is understood as a cycloalkyl group connected, as a substituent, via a lower alkylene. The (cycloalkyl)alkyl group and lower alkylene of a (cycloalkyl)alkyl group may be substituted or unsubstituted.

The terms “(heterocycle)alkyl” and “(heterocyclyl)alkyl” are understood as a heterocycle group connected, as a substituent, via a lower alkylene. The heterocycle group and the lower alkylene of a (heterocycle)alkyl group may be substituted or unsubstituted.

The term “arylalkyl” is intended to mean an aryl group connected, as a substituent, via a lower alkylene, each as defined herein. The aryl group and lower alkylene of a arylalky may be substituted or unsubstituted. Examples include benzyl, substituted benzyl, 2-phenylethyl, 3-phenylpropyl, and naphthylalkyl.

The term “heteroarylalkyl” is understood as heteroaryl groups connected, as substituents, via a lower alkylene, each as defined herein. The heteroaryl and lower alkylene of a heteroarylalkyl group may be substituted or unsubstituted. Examples include 2-thienylmethyl, 3-thienylmethyl, furylmethyl, thienylethyl, pyrrolylalkyl, pyridylalkyl, isoxazolylalkyl, imidazolylalkyl, and their substituted as well as benzo-fused analogs.

The term “halogen atom,” as used herein, means any one of the radio-stable atoms of column 7 of the Periodic Table of the Elements, i.e., fluorine, chlorine, bromine, or iodine, with fluroine and chlorine being preferred.

The terms “protecting group moiety” and “protecting group moieties” as used herein refer to any atom or group of atoms that is added to a molecule in order to prevent existing groups in the molecule from undergoing unwanted chemical reactions. Examples of protecting group moieties are described in T. W. Greene and P. G. M. Wuts, Protective Groups in Organic Synthesis, 3. Ed. John Wiley & Sons, 1999, and in J. F. W. McOmie, Protective Groups in Organic Chemistry Plenum Press, 1973, both of which are hereby incorporated by reference. The protecting group moiety may be chosen in such a way, that they are stable to the reaction conditions applied and readily removed at a convenient stage using methodology known from the art. A non-limiting list of protecting groups include benzyl; substituted benzyl; alkylcarbonyls (e.g., t-butoxycarbonyl (BOC)); arylalkylcarbonyls (e.g., benzyloxycarbonyl, benzoyl); substituted methyl ether (e.g. methoxymethyl ether); substituted ethyl ether; a substituted benzyl ether; tetrahydropyranyl ether; silyl ethers (e.g., trimethylsilyl, triethylsilyl, triisopropylsilyl, t-butyldimethylsilyl, or t-butyldiphenylsilyl); esters (e.g. benzoate ester); carbonates (e.g. methoxymethylcarbonate); acyclic ketal (e.g. dimethyl acetal); cyclic ketals (e.g., 1,3-dioxane or 1,3-dioxolanes); and cyclic dithioketals (e.g., 1,3-dithiane or 1,3-dithiolane). As used herein, any “PG” group(s) such as, without limitation, PG1 and PG₂ represent a protecting group moiety.

The term “ether” as used herein refers to the refers to a chemical moiety with the formula R—O—R′ where R and or R′ is a un-substituted or substituted alkyl group.

The term “ester” refers to a chemical moiety with formula —(R)ₙ—COOR′, where R and R′ are independently selected from the group consisting of alkyl, cycloalkyl, aryl, heteroaryl (bonded through a ring carbon) and heteroalicyclic (bonded through a ring carbon), and where n is 0 or 1.

An “amide” is a chemical moiety with formula —X—C(O)NHR′ or —X—NHC(O)R′, where X is a straight or branched chain or cyclic or heterocyclic moiety, where R and R′ are independently selected from the group consisting of alkyl, cycloalkyl, aryl, heteroaryl (bonded through a ring carbon) and heteroalicyclic (bonded through a ring carbon), and where n is 0 or 1. An amide may be derived by attachment of an amino acid or a peptide molecule attached to a molecule of the present invention, thereby forming a prodrug.

The term “metabolite” refers to a compound to which KW-3902 is converted within the cells of a mammal. The pharmaceutical compositions discussed herein may include a metabolite of KW-3902 instead of KW-3902. The scope of the methods discussed herein includes those instances where KW-3902 is administered to the subject, yet the metabolite is the bioactive entity.

The terms “pure,” “purified,” “substantially purified,” and “isolated” as used herein refer to the compound of the embodiment being free of other, dissimilar compounds with which the compound, if found in its natural state, would be associated in its natural state. In certain embodiments described as “pure,” “purified,” “substantially purified,” or “isolated” herein, the compound may comprise at least 0.5%, 1%, 5%, 10%, or 20%, and most preferably at least 50% or 75% of the mass, by weight, of a given sample.

The terms “derivative,” “variant,” or other similar term refers to a compound that is an analog of the other compound.

### Synthesis of NACA (3-Noradamantane Carboxylic Acid) and NACA Derivatives

Compounds of Formula (I), (II), (III) and (IV) can be readily synthesized using commercially available starting compounds and routine protocols. Synthesis of compounds (I), (II), (III), and (IV) can utilize noradamantane carboxylic acid (NACA) derivatives which can be used to form reactive noradamantane derivatives, which in turn can be prepared from commercially available starting compounds using routine protocols. A synthesis route for an exemplary NACA compound and a corresponding reactive noradamantane derivative is shown below in Scheme 1.

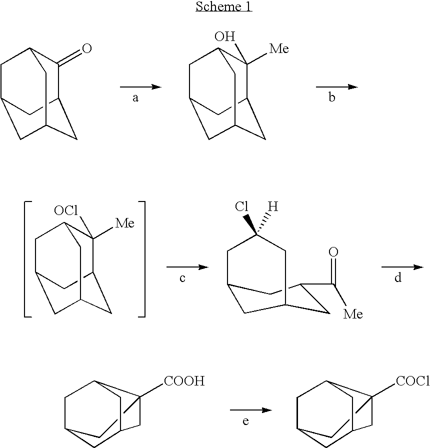

Referring to Scheme 1, in step (a) commercially available 2-adamantanone (Acros Organics, Geel, Belgium, Cat. No. 29250) can be converted to 2-methyl adamantanol using a Grignard reaction. For example, 2-adamantanone can be reacted with a Grignard reagent, such as MeMgBr, MeMgI, or the like in the presence of an appropriate solvent. The reaction can be carried out in several reaction solvents such as benzene, toluene, heptane, hexane, dichloromethane and, ethers such as dioxane, tetrahydrofuran, etc., which can be used alone or in combination. Preferably, the reaction is carried out in the presence of tetrahydrofuran. To this, a saturated aqueous NH₄Cl solution can be added, followed by 6N HCl and ethyl acetate (EtOAc). The EtOAc layer can be washed, and MgSO₄ can be added to dehydrate and concentrate the 2-methyl-adamantanol product.

In steps (b) and (c), the ring structure can be opened by reacting the adamantanol with a halogenating agent, such as NaOCl, NaOBr, NCS, or NBS, in the presence of an appropriate solvent, such as CHCl₃ or CCl₄. In some embodiments, acetic acid can be added in a dropwise manner to a reaction mixture of the adamantanol compound, the halogenating agent, and CCl₄. This forms the intermediate 2-methyl-2-adamantylhypochlorite. Preferably, the reaction is carried out at 2° C. (step (b)). The reaction can proceed and the organic layers can be washed in alkaline solution, such as NaHCO₃ followed by washes with water (step (c)). MgSO₄ can be added to dehydrate the chlorinated derivative. The chlorinated derivative can be heated to 70-100° C. to open the ring structure. Following cooling, the open-ringed adamantanone derivates can be concentrated.

In step (d), the ring structure can be closed again in the presence of a basic solution, such as KOH and a lower alcohol, such as methanol to yield an acetylated noradamantane derivative. The acetylated noradamantane derivative can be converted to a noradamantane-carboxylic acid derivative with Br₂ and NaOH, or another halogen in the presence of a base.

In step (e), the noradamantane carboxylic acid derivative can be converted to reactive derivative, such as an acid chloride, an ester of H-hydroxysuccinimide, a mixed anhydride, a symmetrical anhydride and the like. In some embodiments, the carboxylic acid derivatives from step (d) can be treated with thionyl chloride to generate the acid chloride. Step (e) can be carried out in a variety of solvents, including but not limited to pyridine, dichloroethane, methylene chloride, toluene, chloroform and the like. In some embodiments, the reaction can be carried out between 15° C. and 30° C.

In some embodiments, the noradamantyl groups of KW-3902 or metabolites or prodrugs thereof can be linked to a polar or charged moiety, and used to generate KW-3902 derivative with a polar or charged moiety linked to the noradamantyl group exemplified in Formulas (III) and (IV). In an analogous pathway shown in Scheme 1, the following noradamantyl reactive derivatives can be created starting from commercially available reagents, for use in the synthesis of compounds of Formula (III) and Formula (IV):

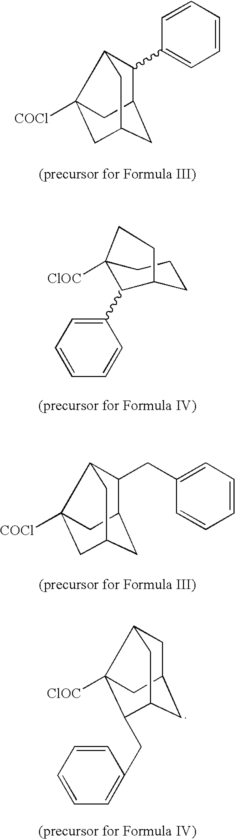

The starting compounds of Formula (III) and Formula (IV) shown above can be synthesized from commercially available 2-adamantanone (Acros Organics, Geel, Belgium, Cat. No. 29250) through the benzyl and phenyl adamantanone intermediates shown below:

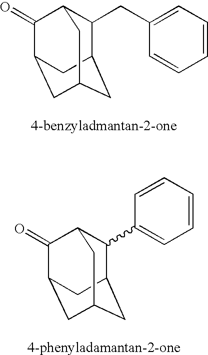

The benzyl and phenyl adamantanone derivatives shown above can be produced using the method described by D. A. Lightner et al. (1987) J. Org. Chem. 52:4171-4175, the disclosure of which is hereby expressly incorporated by reference in its entirety, including any drawings. Isomers may be searated as required using standard chromatographic techniques. The synthesis of exemplary phenyl and benzyl adamantanone derivates is shown in Scheme 2 and Scheme 3, respectively, below.

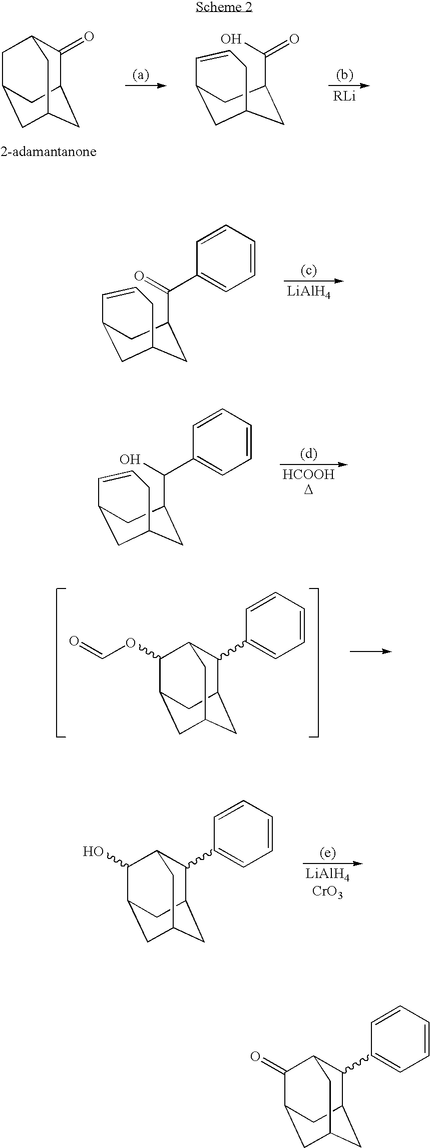

Optionally, the different enantiomers of the adamantanone derivatives can be resolved by crystallization with dehydroabietylarnine. Preparative GC can be used to separate the epimeres as described in Lightner et al. The adamantanone derivatives above can be converted to reactive adamantanane derivatives. Exemplary pathways for the generation of the adamantanane reactive derivatives with benzyl or phenyl substitutions as shown above are set forth in Schemes 3 and 4 below.

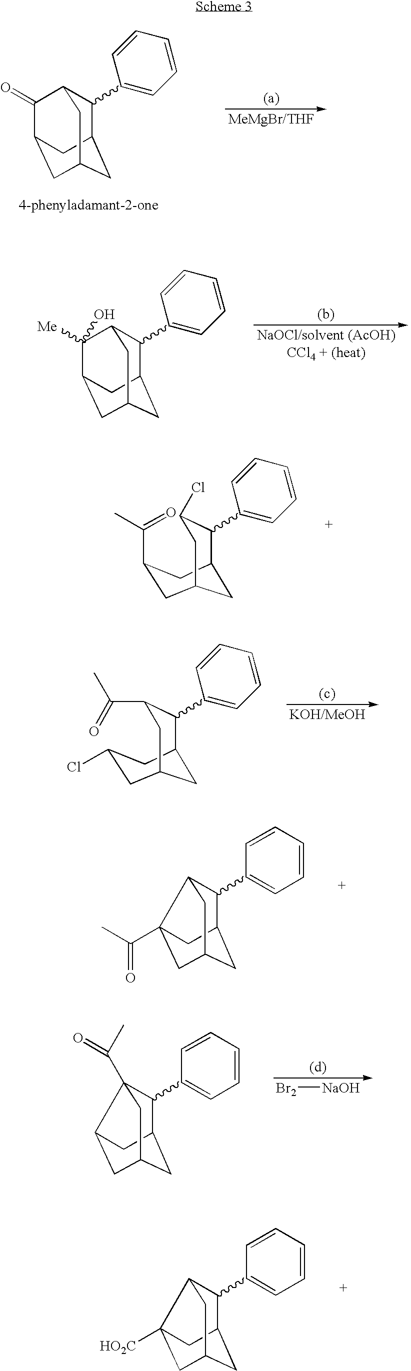

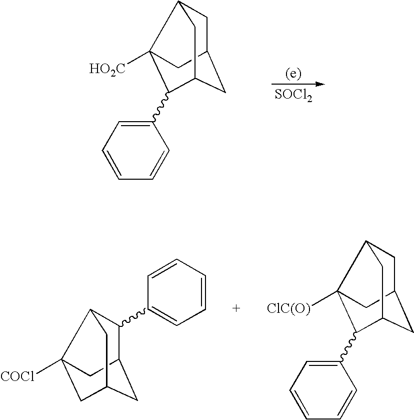

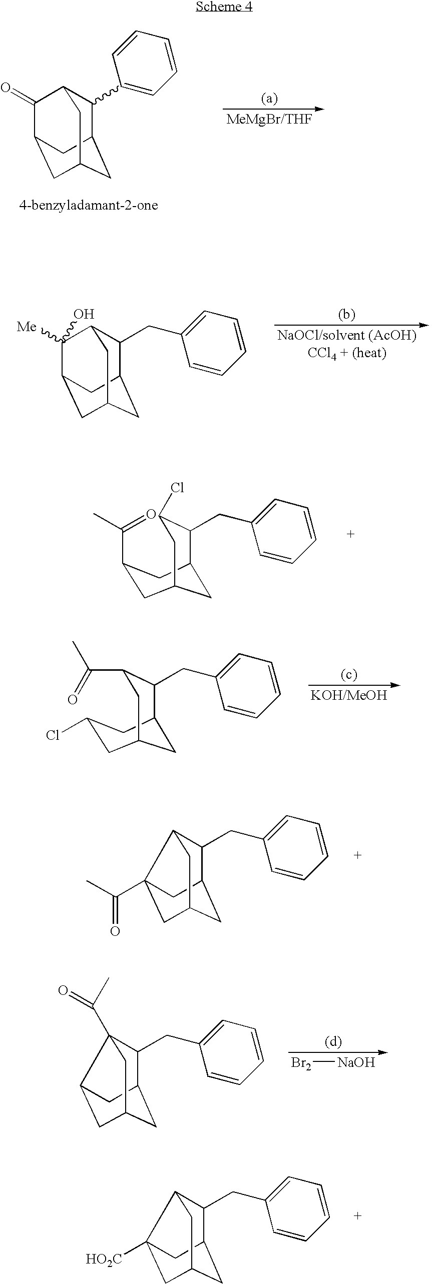

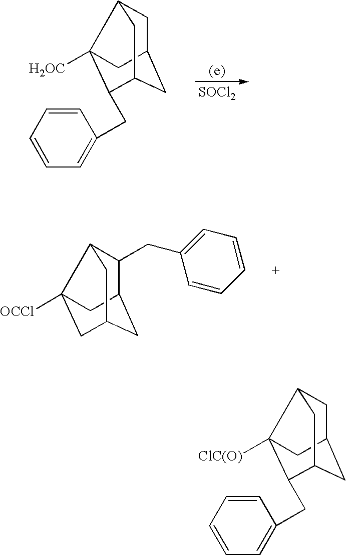

Referring to Schemes 3 and 4, the reactions set forth in steps (a) through (e) are analogous to those described in connection with Scheme 1.

### 1,3-Disubstituted 5,6-Diaminouracils

To produce the compounds of Formulas (I), (II), (III), and (IV) the reactive noradamantane derivatives described above can be combined with a diaminouracil derivative, such as the compound below:

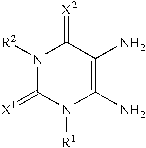

1,3-disubstituted 5, 6-diaminouracils can be prepared by treating the corresponding symmetrically or unsymmetrically substituted urea with cyanoacetic acid, followed by nitrosation and reduction. See, e.g., J. Org. Chem. (1951) 16: 1879; Can. J. Chem. (1968) 46:3413, the entire disclosures of which are each hereby expressly incorporated by reference in their entireties including any figures. Each of R¹ and R² can independently represent hydrogen, lower alkyl, allyl, propargyl, or hydroxy-substituted, oxo-substituted or unsubstituted lower alkyl. Each of X¹ and X² independently represents oxygen or sulfur. The compounds above can be obtained by methods described, for example, in Japanese Published Unexamined Patent Application No. 79296/79, and Japanese Published Unexamined Patent Application No. 42383/84, the entire disclosures of which are each hereby expressly incorporated by reference in its entirety including any figures.

In some preferred embodiments, the disubstituted diaminouracil has the structure

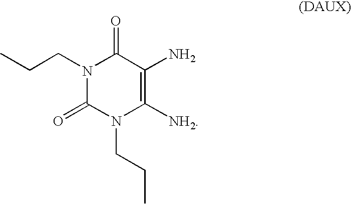

In some embodiments, the following disubstituted diaminouracil compounds can be used in the synthesis of compounds of Formula (I) or Formula (II), having charged or polar moieties linked to the xanthine rings, as described below.

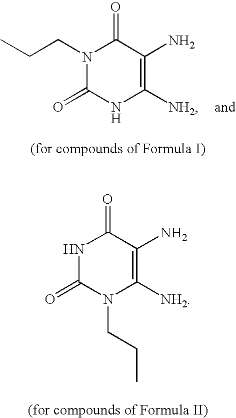

The diaminouracils can be prepared, for example, by the pathways set forth in Scheme 5 and Scheme 6, as well as in J. Org. Chem. (1951) 16: 1879; Can. J. Chem. (1968) 46: 3413, Japanese Published Unexamined Patent Application No. 79296/79, and Japanese Published Unexamined Patent Application No. 42383/84, the entire disclosures of which are each hereby expressly incorporated by reference in its entirety including any figures.

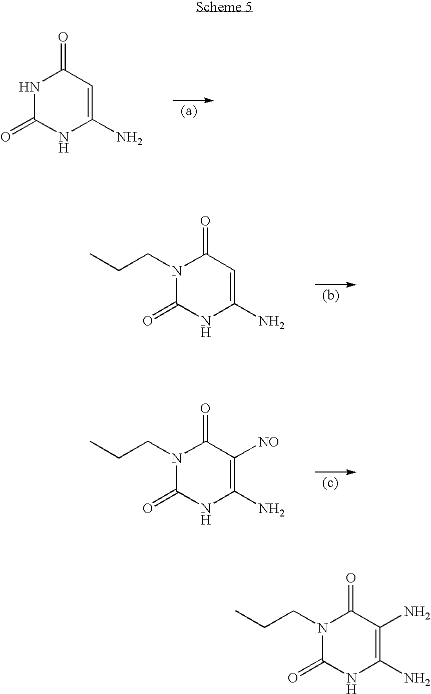

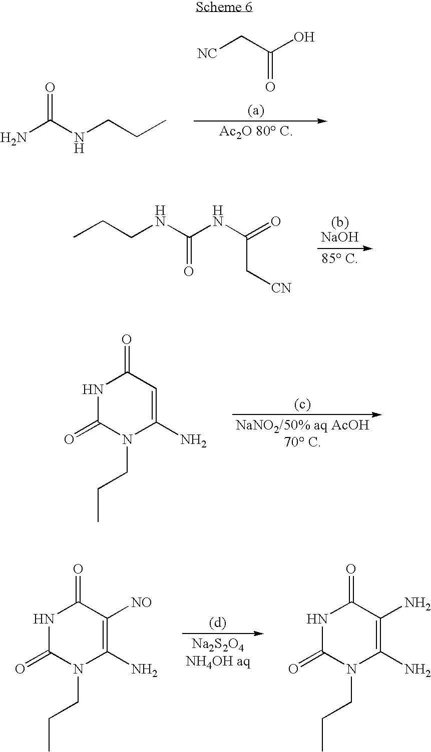

Referring to Scheme 5, in step (a), 6-aminouracil (Sigma Aldrich Cat. NO. 09630, St. Louis, Mo.) is reacted with (NH₄)₂SO₄ in the presence of propyl iodide and aqueous Na₂S₂O₃ to alkylate the compound at the N-3 position. In step (b), 3-propyl, 6-aminouracil is treated with NaNO₂ in an acidic solution, such as aqueous acetic acid in the presence of heat. In step (c) the nitroso group is reduced by treating the compound from step (b) with sodium dithionate (12.5%) in a base, such as aqueous NH₄OH (54%).

Referring to Scheme 6, in step (a), 1-propylurea is reacted with cyanoacetic acid in the presence of acetic anhydride and heat to yield the compound 1-(2-cyanoacetyl)-3-propylurea. In step (b), 1-(2-cyanoacetyl)-3-propylurea is treated with base in the presence of heat to yield 1-propyl 6-aminouracil. In step (c) the 1-propyl 6-aminouracil is treated with NaNO₂ in an acidic solution, such as aqueous acetic acid, in the presence of heat. In step (d) the nitroso group is reduced by treating the compound from step (c) with sodium dithionate (12.5%) in a base, such as aqueous NH₄OH (54%). See, e.g., Beauglehole, A., et al. (2000) 43:4973, the entire disclosure of which is hereby incorporated by reference in its entirety.

### Synthesis of Compounds of Formula (I) and (II)—Substitutions on the Xanthine Ring

The substituted diaminouracils described in the previous section can be used to generate compounds of Formula (I) and Formula (II), including but not limited to compounds Ia and IIa. An exemplary pathway for the synthesis of a compound of Formula (I), e.g., Compound Ia, is depicted in Scheme 7, below.

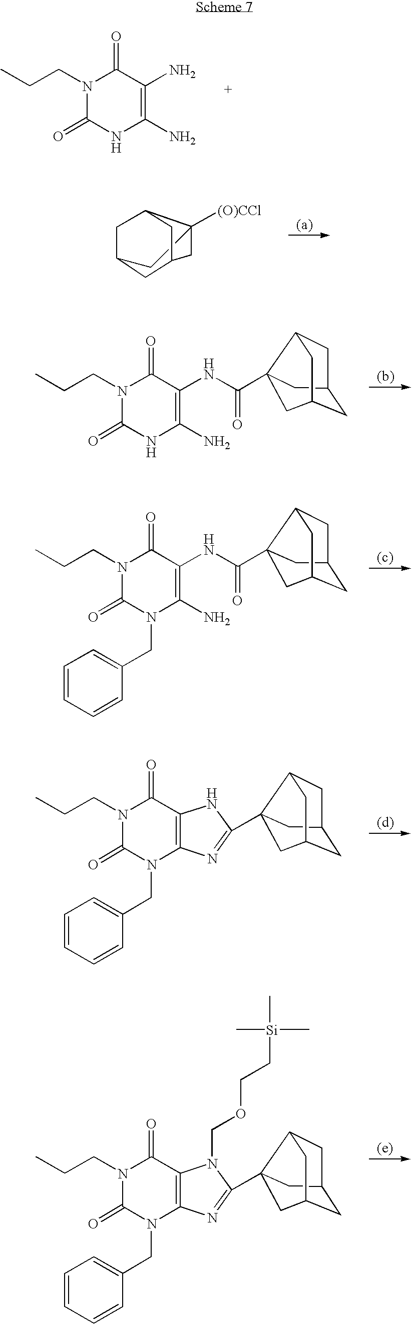

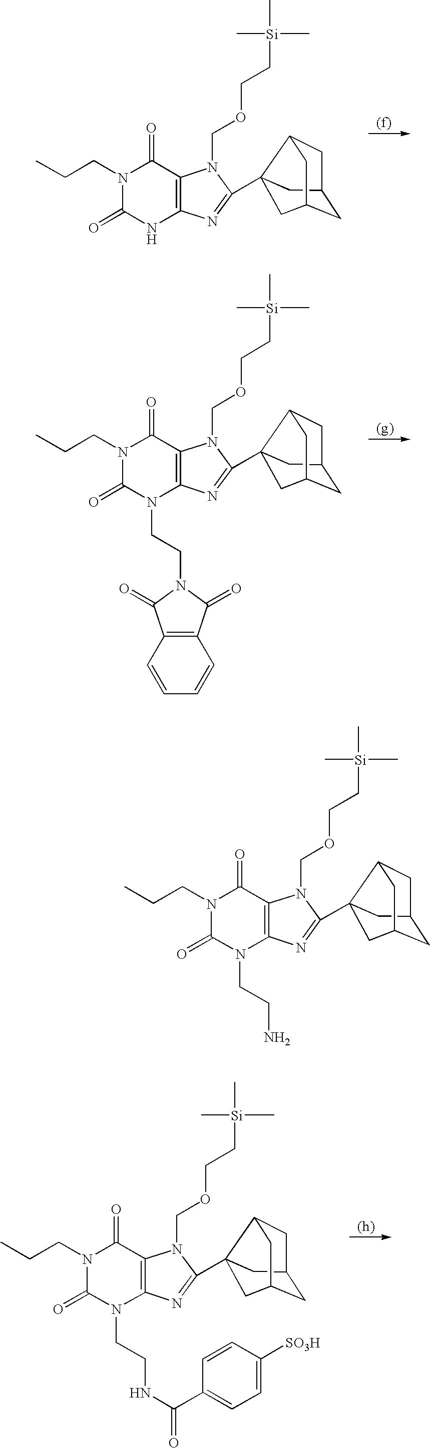

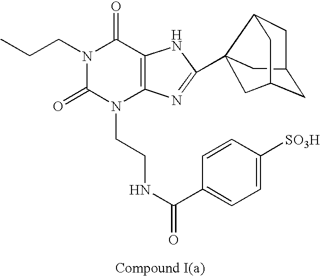

In step (a), the reactive noradamantane derivative is coupled to the diaminouracil derivative at the N-5 position to form the amide under condensation reaction conditions generally used in peptide chemistry. For example, the reaction can be carried out in the presence of an additive or a base. Exemplary solvents for the reaction can include halogenated hydrocarbons such as methylene chloride, chloroform and ethylene dichloride; ethers such as dioxane and tetrahydrofuran; dimethylformamide and dimethylsulfoxide, water, and the like. Exemplary additives include but are not limited to 1-hydroxybenzotriazole and the like. Exemplary bases include pyridine, triethylamine, 4-dimethylaminopyridine, N-methylmorpholine and the like. The reaction can be carried out between about −80° C. to about 50° C. over anywhere between about 30 minutes to about 24 hours.

In step (b), a protective group can be added selectively to the N-1 position of the xanthine ring. For example, benzylbromide can be reacted with N-(4-amino-2,6-dioxo-1-propyl-1,2,3,6-tetrahydropyrimidin-5-yl)(3-noradamantane)carboxamide in the presence of a base such as potassium carbonate, sodium carbonate, or the like. The reaction can proceed in a variety of solvents, including dimethylformamide and the like.

In step (c), the xanthine ring can be formed by treating the alkylated compound with a base, a dehydrating agent, or heat. For example, in some embodiments, the alkylated compound can be treated with a base such as an alkali metal hydroxide such as sodium hydroxide, potassium hydroxide, or the like, or an alkaline metal earth metal hydroxide such as calcium hydroxide. Reaction solvents useful in forming the xanthine ring can include water, a lower alcohol such as methanol or ethanol, an ether such as dioxane or tetrahydrofuran, dimethylformamide, dimethylsulfoxide or the like, or any combination thereof. In some embodiments, the reaction can be carried out at about 0° C. to 180° C. over anywhere between about 30 minutes to about 5 hours. Exemplary bases useful in step (c) reaction include alkali metal hydroxides such as sodium hydroxide, potassium hydroxide, etc. The reaction solvent can be water, lower alcohols such as methanol, ethanol, etc., ethers such as dioxane, tetrahydrofuran, etc., dimethylformamide, dimethylsulfoxide, etc. alone or in combination. The reaction can be carried out from room temperature to 180° C. and is usually completed for about 10 minutes to about 6 hours.

Exemplary dehydrating agents for use in closing the ring structure as set forth in step (c) include thionyl halides such as thionyl chloride, etc., and phosphorous oxyhalides such as phosphorous oxychloride, etc. The reaction can be carried out at a temperature from room temperature to 180° C. without any solvent, or in a solvent that is inert to the reaction, such as halogenohydrocarbons such as methylene chloride, chloroform, dichloroethane, etc., dimethylformamide, dimethylsulfoxide, etc. and can proceed for about 0.5 to 12 hours.

Alternatively, the ring can be closed by heating at a temperature of 50° C. to 200° C. in a polar solvent such as dimethylsulfoxide, dimethylformamide, or the like.

In step (d), the N-9 position of the xanthine ring can be protected. For example, the copound from step (c) can be reacted with 2-(trimethylsilyl)ethyoxymethyl chloride (Sigma Aldrich Cat. No. 92749). The reaction can proceed under standard reaction conditions, for example in the presence of a base such as potassium carbonate, sodium carbonate or the like. The reaction can be carried out in a solvent such as dimethylformamide, dimethylsulfoxide or the like.

In step (c), the protecting group is removed from the N-1 position using standard reaction conditions. For example, the protecting group can be removed in the presence of hydrogen gas or ammonium formate, using 10% Pd/C (Palladium on activated carbon) or palladium hydroxide on carbon as a catalyst in a solvent such as lower alcohols, e.g., methanol or ethanol.

In step (f), N-1 position can be reacted with a compound such as N-(2-bromoethyl)phthalimide (Sigma Aldrich Cat. No. 16170) in the presence of a base such as potassium carbonate, sodium carbonate, cesium carbonate, sodium hydride, potassium tert-butoxide or the like to eventually form a reactive propyl amine group. The reaction can proceed in the presence of a solvent such as dimethylformamide, dimethylsulfoxide or the like.

In step (g), the phthalimide group is removed to yield an ethyl amine group at the N-1 position of the xanthine ring. This can proceed, for example, by treating the compound from step (f) with hydrazine monohydrate. The reaction can proceed in the presence of a solvent such as dimethylformamide or the like.

The resulting reactive amine group on the compound from step (g) 3-(2-aminoethyl)-8-(3-noradamantyl)-1-propyl-7-((2-(trimethylsilyl)ethoxy)methyl)-1H-purine-2,6(3H,7H-dione can be derivatized, for example, with a sulfobenzene moiety or the like. For example, the product of step (g) can be reacted with, for example, 4-sulfobenzoic acid monopotassium salt (Cole-Parner Cat. No. EW-88357-07, Vernon Hills, Ill.), or a similar reactive polar or charged moiety. The reaction can proceed, for example, in a solvent such as ethylene dichloride in an alcohol, such as methanol.

In step (i), the N-9 protective group can be removed to yield the compound Ia. The deprotection can proceed, for example, in an acid such as hydrochloric acid or the like, in a lower alcohol, such as ethanol methanol or the like.

### Synthesis of Compounds of Formula (II)

An exemplary pathway for the synthesis of Formula (II), e.g., Compound IIa, is depicted in Scheme 8:

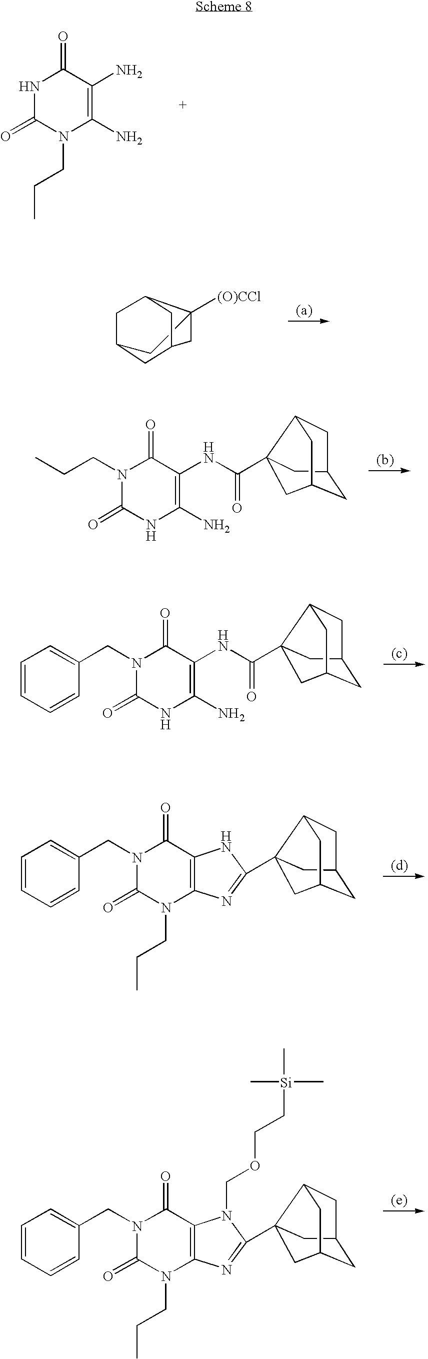

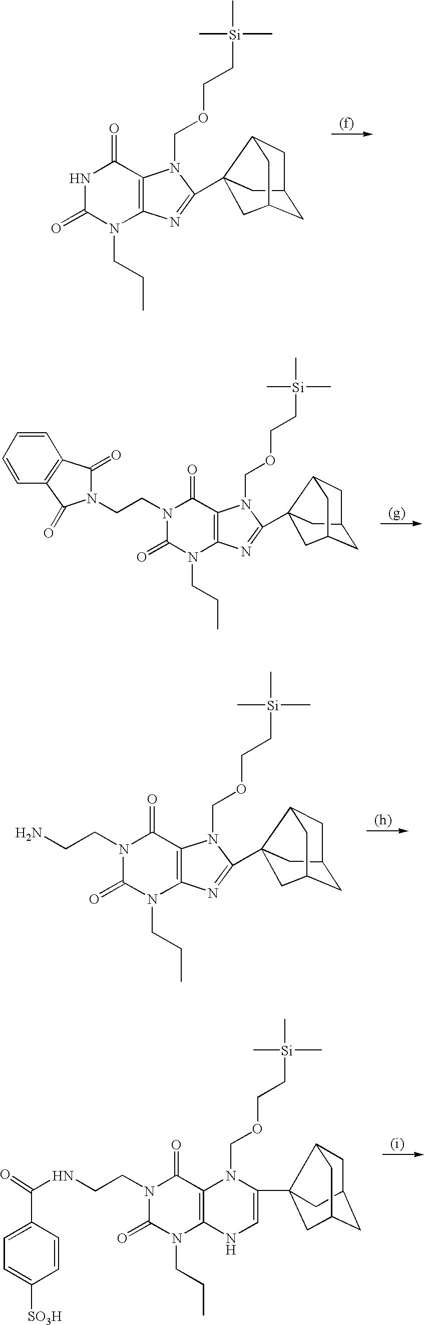

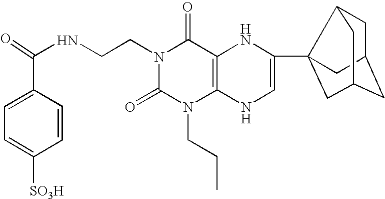

Referring to Scheme 8, the reactions set forth in steps (a) through (i) are analogous to those described in relation to Scheme 7. The pathway shown in Scheme 8 will yield compounds of Formula II, such as Compound Ia.

### Synthesis of Compounds of Formula (II) and (IV)

Scheme 9 depicts an exemplary pathway for synthesizing xanthine derivatives that are substituted on the noradamantyl group, such as compounds of Formula (III).

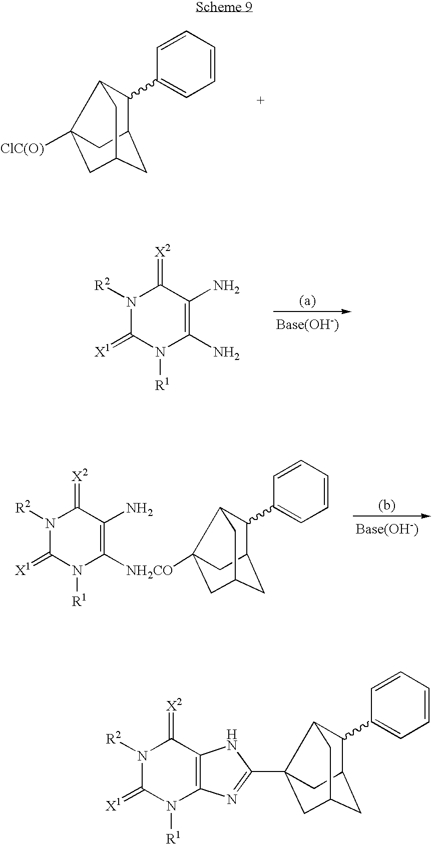

In step (a), a reactive noradamantane derivative such as those generated in schemes 3 and 4 is joined to the noradamantyl derivative using standard conditions for condensation, such as those set forth in Schemes 7 and 8. The ring structure of the intermediate compound can be closed to yield a compound of Formula (III) either in the presence of a base, a dehydrating agent or heat, as described above in Schemes 7 and 8.

By analogy, compounds of Formula (IV) can be produced using similar reaction conditions with noradamantyl derivatives such as the compounds shown below, and the like.

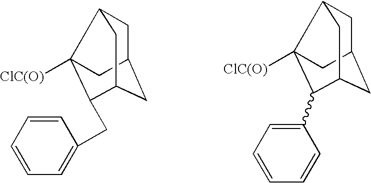

### Derivatization with Polar Groups

Polar groups such as SO₃, SO₂H, PO₃, PO₂H, NO₃, NO₂H, CF₃, CH₂F, CHF₂, cyano, isocyano, amide, guanidinium, and NH₂ can be added to compounds yielded by the procedures described above using standard reaction conditions. For example, sulfonation of compounds from Scheme 9 can be accomplished by combining the compounds generated from step (b) with H₂SO₄, SO₃ in the presence of heat. Similarly, nitration of the compounds can be accomplished by combining compounds of Scheme 9 with HNO₃, H₂SO₄ in the presence of heat. For example, sulfonation of compounds of Formula (II) is shown below.

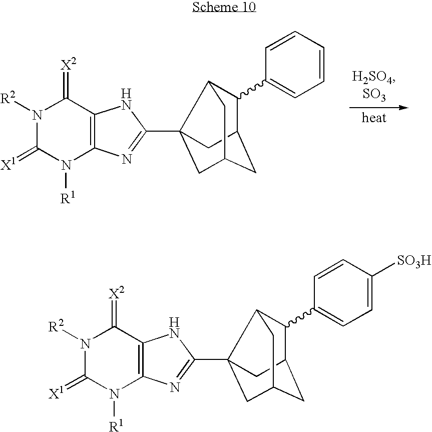

Preferred embodiments provide the following compounds:

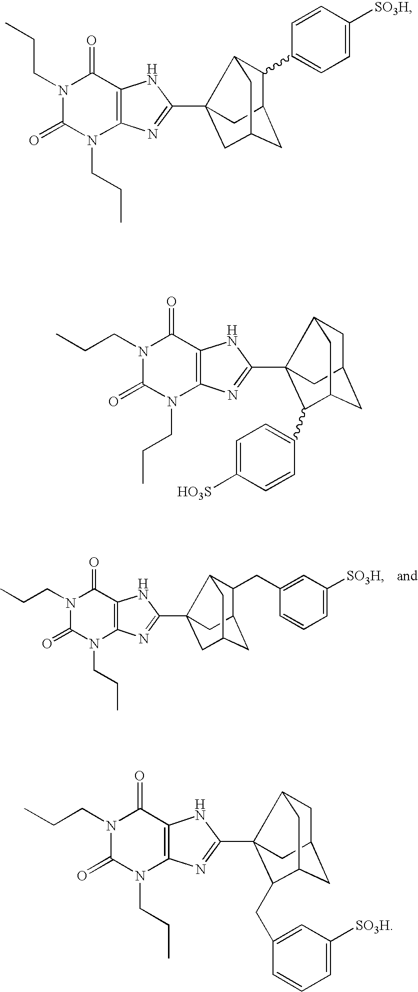

### KW-3902 Derivatives with Ether-Linked Substitutions

Also provided herein are methods for synthesizing derivatives of KW-3902 wherein the noradamantyl group of KW-3902 is substituted via an ether linkage, such as in compounds of Formula (V) and Formula (VI):

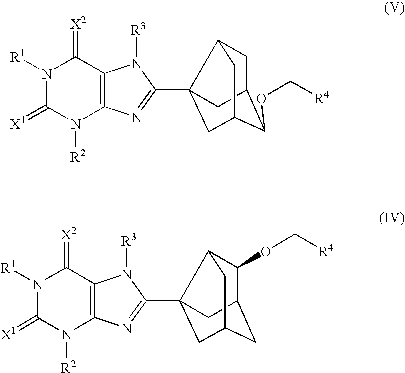

Conventional reactions can be used to synthesize the compounds of Formula (V) and Formula (VI) from known starting compounds, such as those described in U.S. Pat. No. 5,290,782 issued Mar. 1, 1994, the entire contents of which is hereby expressly incorporated by reference in its entirety. The synthesis of compounds of Formula (V) and Formula (VI) are set forth in Schemes 11 and 12, below.

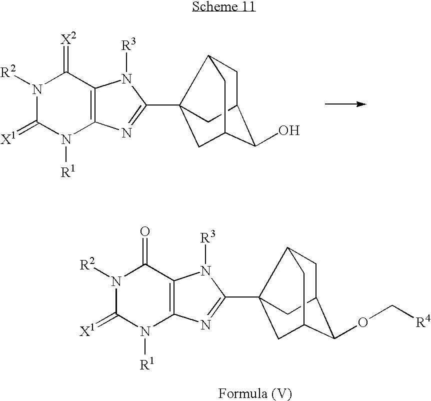

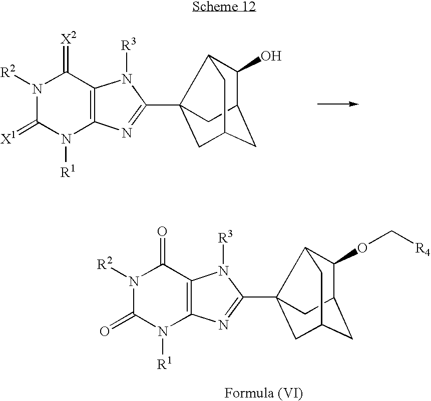

In preferred embodiments, the compounds below are used to produce compounds of Formula (V) and Formula (VI):

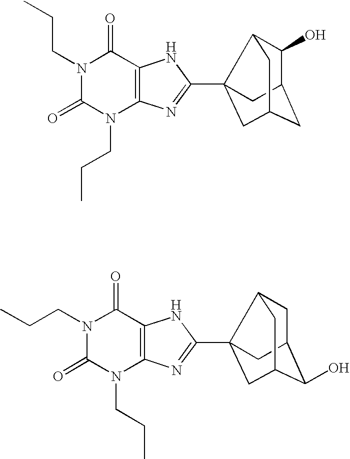

An exemplary compound of Formula (VI) is shown below: 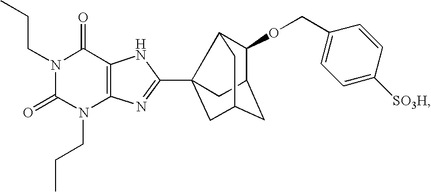
An exemplary compound of Formula (V) is shown below:

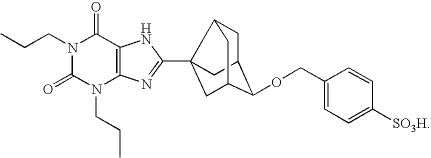

Also provided herein are compounds of the structure

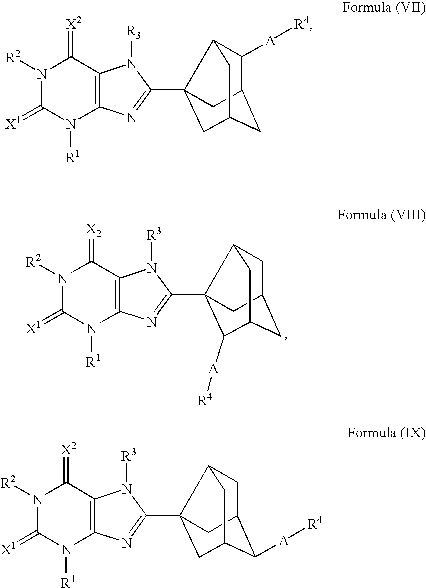

wherein A is any suitable linking group, such as a substituted or unsubstituted, branched or unbranched alkyl, alkenyl, alkynyl, ester, ether, thioether, or amide linking group. In some embodiments, the linking group A may comprise a combination of carbon chain moieties (e.g., —CH₂— or —CH═ or carbonyl) with ester, ether, thioether, or amide linkages. The number of chain moieties can range from 0, 1, 2, 3, 4, 5, or more up to 10, 12, 14, 16, or 20 (or even more) chain moieties or carbon residues. R⁴ is defined as above.

### Pharmaceutical Compositions

Some embodiments provided herein relate to pharmaceutical compositions comprising a KW-3902 derivative of Formula (I), Formula (II), Formula (III), Formula (IV), Formula (V), or Formula (VI) as described above, and a physiologically acceptable carrier, diluent, or excipient, or a combination thereof.

The term “pharmaceutical composition” refers to a mixture of a compound Of the invention with other chemical components, such as diluents or carriers. The pharmaceutical composition facilitates administration of the compound to an organism. Multiple techniques of administering a compound exist in the art including, but not limited to, oral, injection, aerosol, parenteral, and topical administration. Pharmaceutical compositions can also be obtained by reacting compounds with inorganic or organic acids such as hydrochloric acid, hydrobromic acid, sulfuric acid, nitric acid, phosphoric acid, methanesulfonic acid, ethanesulfonic acid, p-toluenesulfonic acid, salicylic acid and the like.

The term “carrier” defines a chemical compound that facilitates the incorporation of a compound into cells or tissues. For example dimethyl sulfoxide (DMSO) is a commonly utilized carrier as it facilitates the uptake of many organic compounds into the cells or tissues of an organism.

The term “diluent” defines chemical compounds diluted in water that will dissolve the compound of interest as well as stabilize the biologically active form of the compound. Salts dissolved in buffered solutions are utilized as diluents in the art. One commonly used buffered solution is phosphate buffered saline because it mimics the salt conditions of human blood. Since buffer salts can control the pH of a solution at low concentrations, a buffered diluent rarely modifies the biological activity of a compound.

The term “physiologically acceptable” defines a carrier or diluent that does not abrogate the biological activity and properties of the compound.

The pharmaceutical compositions described herein can be administered to a human subject per se, or in pharmaceutical compositions where they are mixed with other active ingredients, as in combination therapy, or suitable carriers or excipient(s). Techniques for formulation and administration of the compounds of the instant application may be found in “Remington's Pharmaceutical Sciences,” Mack Publishing Co., Easton, Pa., 18th edition, 1990.

Suitable routes of administration may, for example, include oral, rectal, transmucosal, or intestinal administration; parenteral delivery, including intramuscular, subcutaneous, intravenous, intramedullary injections, as well as intrathecal, direct intraventricular, intraperitoneal, intranasal, or intraocular injections.

Alternatively, one may administer the compound in a local rather than systemic manner, for example, via injection of the compound directly in the renal or cardiac area, often in a depot or sustained release formulation. Furthermore, one may administer the drug in a targeted drug delivery system, for example, in a liposome coated with a tissue-specific antibody. The liposomes will be targeted to and taken up selectively by the organ.

The pharmaceutical compositions disclosed herein may be manufactured in a manner that is itself known, e.g., by means of conventional mixing, dissolving, granulating, dragee-making, levigating, emulsifying, encapsulating, entrapping or tabeleting processes.

Pharmaceutical compositions for use in accordance with the embodiments described herein thus may be formulated in conventional manner using one or more physiologically acceptable carriers comprising excipients and auxiliaries which facilitate processing of the active compounds into preparations which can be used pharmaceutically. Proper formulation is dependent upon the route of administration chosen. Any of the well-known techniques, carriers, and excipients may be used as suitable and as understood in the art; e.g., in Remington's Pharmaceutical Sciences, above.

For injection, the agents of the invention may be formulated in aqueous solutions or lipid emulsions, preferably in physiologically compatible buffers such as Hanks's solution, Ringer's solution, or physiological saline buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a subject to be treated. Pharmaceutical preparations for oral use can be obtained by mixing one or more solid excipient with pharmaceutical combination of the invention, optionally grinding the resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethyl cellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Furthermore, the formulations of the present invention may be coated with enteric polymers. All formulations for oral administration should be in dosages suitable for such administration.

For buccal administration, the compositions may take the form of tablets or lozenges formulated in conventional manner.

For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

A pharmaceutical carrier for the hydrophobic compounds described herein is a cosolvent system comprising benzyl alcohol, a nonpolar surfactant, a water-miscible organic polymer, and an aqueous phase. A common cosolvent system used is the VPD co-solvent system, which is a solution of 3% w/v benzyl alcohol, 8% w/v of the nonpolar surfactant POLYSORBATE 80™, and 65% w/v polyethylene glycol 300, made up to volume in absolute ethanol. Naturally, the proportions of a co-solvent system may be varied considerably without destroying its solubility and toxicity characteristics. Furthermore, the identity of the co-solvent components may be varied: for example, other low-toxicity nonpolar surfactants may be used instead of POLYSORBATE 80™; the fraction size of polyethylene glycol may be varied; other biocompatible polymers may replace polyethylene glycol, e.g., polyvinyl pyrrolidone; and other sugars or polysaccharides may substitute for dextrose.

Alternatively, other delivery systems for hydrophobic pharmaceutical compounds may be employed. Liposomes and emulsions are well known examples of delivery vehicles or carriers for hydrophobic drugs. Certain organic solvents such as dimethylsulfoxide also may be employed, although usually at the cost of greater toxicity. Additionally, the compounds may be delivered using a sustained-release system, such as semipermeable matrices of solid hydrophobic polymers containing the therapeutic agent. Various sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

Some emulsions used in solubilizing and delivering the xanthine derivatives described above are discussed in U.S. Pat. No. 6,210,687, and U.S. Patent Application No. 60/674,080, the disclosures of which are each hereby incorporated by reference in their entirety, including any drawings.

Many of the compounds used in the pharmaceutical combinations described herein can be provided as salts with pharmaceutically compatible counterions. Pharmaceutically compatible salts may be formed with many acids, including but not limited to hydrochloric, sulfuric, acetic, lactic, tartaric, malic, succinic, etc. Salts tend to be more soluble in aqueous or other protonic solvents than are the corresponding free acid or base forms.

Pharmaceutical compositions suitable for use in the embodiments described herein include compositions where the active ingredients are contained in an amount effective to achieve its intended purpose. More specifically, a therapeutically effective amount means an amount of compound effective to prevent, alleviate or ameliorate symptoms of disease or prolong the survival of the subject being treated. Determination of a therapeutically effective amount is well within the capability of those skilled in the art, especially in light of the detailed disclosure provided herein.

The exact formulation, route of administration and dosage for the pharmaceutical compositions disclosed herein can be chosen by the individual physician in view of the subject's condition. (See e.g., Fingl et al. 1975, in “The Pharmacological Basis of Therapeutics”, Ch. 1 p. 1). Typically, the dose range of the composition administered to the subject can be from about 0.5 to 1000 mg/kg of the subject's body weight. The dosage may be a single one or a series of two or more given in the course of one or more days, as is needed by the subject.

The daily dosage regimen for an adult human subject may be, for example, an oral dose of between 0.1 mg and 500 mg, preferably between 1 mg and 250 mg, e.g. 5 to 200 mg or an intravenous, subcutaneous, or intramuscular dose of between 0.01 mg and 100 mg, preferably between 0.1 mg and 60 mg, e.g. 1 to 40 mg of the pharmaceutical compositions described herein or a pharmaceutically acceptable salt thereof calculated as the free base, the composition being administered 1 to 4 times per day. Alternatively the compositions described herein may be administered by continuous intravenous infusion, preferably at a dose of up to 400 mg per day. Thus, the total daily dosage by oral administration will be in the range 1 to 2000 mg and the total daily dosage by parenteral administration will be in the range 0.1 to 400 mg. Suitably the compounds will be administered for a period of continuous therapy, for example for a week or more, or for months or years.

Dosage amount and interval may be adjusted individually to provide plasma levels of the active moiety which are sufficient to maintain the modulating effects, or minimal effective concentration (MEC). The MEC will vary for each compound but can be estimated from in vitro data. Dosages necessary to achieve the MEC will depend on individual characteristics and route of administration. However, HPLC assays or bioassays can be used to determine plasma concentrations.

Dosage intervals can also be determined using MEC value. Compositions should be administered using a regimen which maintains plasma levels above the MEC for 10-90% of the time, preferably between 30-90% and most preferably between 50-90%.

In cases of local administration or selective uptake, the effective local concentration of the drug may not be related to plasma concentration.

The amount of composition administered will, of course, be dependent on the subject being treated, on the subject's weight, the severity of the affliction, the manner of administration and the judgment of the prescribing physician.

The compositions may, if desired be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Compositions comprising a compound of the invention formulated in a compatible pharmaceutical carrier may also be prepared, placed in an appropriate container, and labeled for treatment of an indicated condition.

The examples described above are set forth solely to assist in the understanding of the embodiments. Thus, those skilled in the art will appreciate that the methods may provide derivatives of compounds.

### Methods of Using KW-3902 Derivatives

Provided herein are methods of treating subjects using a therapeutically effective amount of the KW-3902 derivatives described above, or a salt, ester, amide, metabolite, or prodrug thereof. Other embodiments relate to inducing diuresis in a subject in need thereof, by identifying a subject in need thereof and providing to the subject a therapeutically effective amount of a compound of Formula (I), (II), (III), (IV), (V) or (VI) or a pharmaceutically acceptable salt, ester, metabolite, or prodrug thereof In some embodiments a non adenosine-modifying diuretic is also provided.

Yet other embodiments relate to maintaining or restoring the diuretic effect of a non-adenosine modifying diuretic in a subject by providing a compound of Formula (I), (II), (III), (IV), (V) or (VI), r or a pharmaceutically acceptable salt, ester, metabolite, or prodrug thereof and a non adenosine-modifying diuretic.

Still other embodiments relate to methods of improving renal function by identifying a subject suffering from impaired creatinine clearance and providing to said subject a therapeutically amount of Formula (I), (II), (III), (IV), (V) or (VI), or a pharmaceutically acceptable salt, ester, metabolite, or prodrug thereof effective to maintain or increase creatinine clearance, and a non adenosine-modifying diuretic.

Other embodiments relate to methods of maintaining renal function by identifying a subject with impaired creatinine clearance and providing to the subject a therapeutically effective amount of Formula (I), (II), (III), (IV), (V) or (VI), or a pharmaceutically acceptable salt, ester, metabolite, or prodrug thereof and a non adenosine-modifying diuretic, thereby slowing or arresting the impairment in creatinine clearance for a period of time.

Other embodiments relate to methods of restoring renal function, by identifying a subject having increased serum creatinine levels and/or decreased creatinine clearance and providing to said subject a therapeutically effective amount of a compound of Formula (I), (II), (III), (IV), (V) or (VI), or a pharmaceutically acceptable salt, ester, metabolite, or prodrug thereof, and a non adenosine-modifying diuretic, thereby decreasing serum creatinine levels and/or slowing or arresting the impairment of creatinine clearance.

Yet other embodiments relate to methods of improving, maintaining, or restoring renal function by identifying a subject suffering from congestive heart failure and renal impairment and providing to the subject a therapeutically effective amount of a compound of Formula (I), (II), (III), (IV), (V) or (VI), or a pharmaceutically acceptable salt, ester, metabolite, or prodrug thereof, and a non adenosine-modifying diuretic.

Still other embodiments relate to methods of improving, maintaining, or restoring renal function in a subject by identifying a subject that is suffering from congestive heart failure who is refractory to standard diuretic therapy and providing to the subject a therapeutically effective amount of a compound of Formula (I), (II), (III), (IV), (V) or (VI), or a pharmaceutically acceptable salt, ester, metabolite, or prodrug thereof, effective to maintain or increase creatinine clearance and a diuretic.

Yet other embodiments relate to methods of treating acute fluid overload in a subject by identifying a subject in need of short-term hospitalization to treat acute fluid overload, hospitalizing said subject, providing the subject with a non adenosine-modifying diuretic and a therapeutically effective amount of a compound of Formula (I), (II), (III), (IV), (V), or (VI) or a pharmaceutically acceptable salt, ester, metabolite, or prodrug thereof effective to accelerate removal of excess fluid from the subject compared to diuretic therapy alone.

Still other embodiments relate to methods of improving, maintaining, or restoring renal function in subjects with stable congestive heart failure taking chronic diuretics by identifying a subject with stable congestive heart failure taking chronic diuretics and providing to the subject a therapeutically effective amount of a compound of Formula (I), (II), (III), (IV), (V) or (VI), or a pharmaceutically acceptable salt, ester, metabolite, or prodrug thereof in about four day to about monthly intervals, wherein the subject simultaneously continues the chronic diuretic therapy throughout the course of treatment with the compound of Formula (I), (II), (III), (IV), (V), or (VI).

Some embodiments provide methods of improving diuresis while maintaining renal function in individuals with fluid overload using a therapeutically effective amount of a compound of Formula (I), (II), (III), (IV), (V), or (VI), or a salt, ester, amide, metabolite, or prodrug thereof, and a non-adenosine modifying diuretic.

The term “therapeutically effective amount” as used herein refers to that amount of a composition being administered which will relieve to some extent one or more of the signs or symptoms of the disorder being treated.

In certain embodiments, the subject being treated by the methods described herein suffers from renal impairment. In other embodiments, the subject does not suffer from renal impairment.

Renal function refers to the ability the kidney to excrete waste and maintain a proper chemical balance. Typically, renal function is measured by plasma concentrations of creatinine, urea, and electrolytes to determine renal function. Creatinine is a byproduct of normal muscle metabolism that is produced at a fairly constant rate in the body and normally filtered by the kidneys and excreted in the urine. It will be appreciated that any method known to those skilled in the art for measuring renal function can be used in the methods described herein. For example, serum creatinine levels, urine creatinine levels, and glomerular filtration rate (GFR) can be used to assess renal function.

Normal serum creatinine levels in adult males are generally from about 0.8-1.4 mg/dL. Normal serum creatinine levels in adult females are generally 0.6-1.1 mg/dL. Normal serum creatinine levels in children are generally between about 0.2-1.0 mg/dL. In some embodiments, the subject has elevated serum creatinine levels that are above 2.0 mg/dL, above 3.0 mg/dL, about 4.0 mg/dL, above 5.0 mg/dL, above 6.0 mg/dL, above 7.0 mg/dL, above 8.0 mg/dL, above 9.0 mg/dL, above 10 mg/dL, above 12 mg/dL, above 14 mg/dL, above 16 mg/dL, above 18 mg/dL, above 20 mg/dL, above25 mg/dL, or any number or fraction in between.

In some embodiments, impaired renal function refers to a GFR of less than about 80 mL/min, for example about 20 mL/min, 30 mL/min, 40 mL/min, 50 mL/min, 60 mL/min 70 mL/min or 75 mL/min, or any number in between prior to hospitalization. Accordingly, in some embodiments, the subject exhibits mildly impaired renal function (e.g., a GFR of about 50 to about 80 mL/min). In some embodiments, the subject exhibits moderately impaired renal function (e.g., a GFR of about 30 mL/min to about 50 mL/min). In yet other embodiments, the subject exhibits severely impaired renal function (e.g., a GFR of equal to or less than about 30 mL/min).

In some embodiments, the subject has impaired creatinine clearance. In some embodiments, the subject has elevated serum creatinine levels. In some embodiments, the subjects with impaired creatinine clearance or elevated serum creatinine levels can have from heart failure, such as congestive heart failure, or other maladies that result in fluid overload, without having yet disrupted normal kidney function. In some embodiments, the subject being treated by the methods described herein is refractory to standard diuretic therapy. In other embodiments, the subject is not refractory to standard diuretic therapy.

Other embodiments related to methods of preventing the deterioration of renal function in individuals comprising administering a therapeutically effective amount of KW-3902, or a salt, ester, amide, metabolite, or prodrug thereof, and a non-adenosine modifying diuretic.

In some embodiments, the pharmaceutical compositions can also include a non-adenosine modifying diuretic. In some embodiments, the non-adenosine modifying diuretic is a proximal diuretic, i.e., a diuretic that principally acts on the proximal tubule. Examples of proximal diuretics include, but are not limited to, acetazolamide, methazolamide, and dichlorphenamide. Carbonic anhydrase inhibitors are known to be diuretics that act on the proximal tubule, and are therefore, proximal diuretics. Thus, some embodiments provide compositions that include the combination of a KW-3902 derivative of Formula (I), Formula (II), Formula (III), Formula (IV), Formula (V), Formula (VI) or a pharmaceutically acceptable salt, ester, amide, prodrug or metabolite thereof with a carbonic anhydrase inhibitor. Combinations of KW-3902 derivative of Formula (I), Formula (II), Formula (III), Formula (IV), Formula (V), Formula (VI) or a pharmaceutically acceptable salt, ester, amide, prodrug or metabolite thereof with any proximal diuretic now known or later discovered are within the scope of the embodiments disclosed herein.

In other embodiments, the non-adenosine modifying diuretic is a loop diuretic, i.e., a diuretic that principally acts on the loop of Henle. Examples of loop diuretics include, but are not limited to, furosemide (LASIX®), bumetanide (BUMEX®), and torsemide (TOREM®). Combinations of a KW-3902 derivative of Formula (I), (II), (III), (IV), (V) or (VI) with any loop diuretic now known or later discovered are within the scope of the embodiments disclosed herein.

In yet other embodiments, the non-adenosine modifying diuretic is a distal diuretic, i.e., a diuretic that principally acts on the distal nephron. Examples of distal diuretics include, but are not limited to, metolazone, thiazides and amiloride. Combinations of an KW-3902 derivative of Formula (I), Formula (II), Formula (III), Formula (IV), Formula (V), Formula (VI) or a pharmaceutically acceptable salt, ester, amide, prodrug or metabolite thereof any distal diuretic now known or later discovered are within the scope of the embodiments disclosed herein.

In some embodiments, the compositions provided herein can also include a beta-blocker. A number of beta-blockers are commercially available. These compounds include, but are not limited to, acebutolol hydrochloride, atenolol, betaxolol hydrochloride, bisoprolol fumarate, carteolol hydrochloride, esmolol hydrochloride, metoprolol, metoprolol tartrate, nadolol, penbutolol sulfate, pindolol, propranolol hydrochloride, succinate, and timolol maleate. Beta-blockers, generally, are beta₁ and/or beta₂ adrenergic receptor blocking agents, which decrease the positive chronotropic, positive inotropic, bronchodilator, and vasodilator responses caused by beta-adrenergic receptor agonists. The embodiments disclosed herein include combinations with all beta-blockers now known and all beta-blockers discovered in the future.

In some embodiments, the compositions provided herein can also include an angiotensin converting enzyme inhibitor or an angiotensin II receptor blocker. A number of ACE inhibitors are commercially available. These compounds, whose chemical structure is somewhat similar, include lisinopril, enalapril, quinapril, ramipril, benazepril, captopril, fosinopril, moexipril, trandolapril, and perindopril. ACE inhibitors, generally, are compounds that inhibit the action of angiotensin converting enzyme, which converts angiotensin I to angiotensin II. It will be appreciated that all ACE inhibitors now known and discovered in the future are can be used in the embodiments provided herein.

A number of ARBs are also commercially available or known in the art. These compounds include losartan, irbesartan, candesartan, telmisartan, eposartan, and valsartan. ARBs reduce blood pressure by relaxing blood vessels. This allows better blood flow. ARBs function stems from their ability to block the binding of angiotensin II, which would normally cause vessels to constrict. It will be appreciated that all ARBs now known and discovered in the future can be used in the embodiments provided herein.

In certain embodiments, the subject may be a mammal. The mammal may be selected from the group consisting of mice, rats, rabbits, guinea pigs, dogs, cats, sheep, goats, cows, primates, such as monkeys, chimpanzees, and apes, and humans. In some embodiments, the subject is a human.

In another aspect, the present invention relates to a method of maintaining or restoring the diuretic effect of a non-adenosine modifying diuretic in a subject, while reducing the potential of related adverse events occurring, such as seizures or convulsions, comprising identifying a subject in need thereof, and administering to the subject a therapeutically effective amount of a KW-3902 derivative of Formula (I), Formula (II), Formula (III), Formula (IV), Formula (V), Formula (VI) or a pharmaceutically acceptable salt, ester, amide, prodrug or metabolite thereof. For example some embodiments relate to methods of maintaining or restoring the diueritic effect of a diuretic such as furosemide. In some embodiments, furosemide is administered in a dose of 20 mg, 40 mg, 60 mg, 80 mg, 100 mg, 120 mg, 140 mg, or 160 mg, or higher. The administration may be oral or intravenous. When furosemide is administered intravenous, it may be administered as a single injection or as a continuous infusion. When the administration is through a continuous infusion, the dosage of furosemide may be less than 1 mg per hour, 1 mg per hour, 3 mg per hour, 5 mg per hour, 10 mg per hour, 15 mg per hour, 20 mg per hour, 40 mg per hour, 60 mg per hour, 80 mg per hour, 100 mg per hour, 120 mg per hour, 140 mg per hour, or 160 mg per hour, or higher.

In yet another aspect, the present invention relates to a method of maintaining or restoring renal function in a subject comprising identifying a subject in need thereof, and administering a therapeutically effective amount of a KW-3902 derivative of Formula (I), Formula (II), Formula (III), Formula (IV), Formula (V), Formula (VI) or a pharmaceutically acceptable salt, ester, amide, prodrug or metabolite thereof and a second pharmaceutical composition capable of inducing a diuretic effect.

In the context of the present disclosure, by “maintaining” renal function it is meant that the renal function, as measured by creatinine clearance rate, remains unchanged for a period of time after the start of the therapy. In other words, by “maintaining” renal function it is meant that the rate of renal impairment, i.e., the rate of decrease in the creatinine clearance rate, is slowed or arrested for a period of time, however brief that period may be. By “restoring” renal function it is meant that the renal function, as measured by creatinine clearance rate, has improved, i.e., has become higher, after the start of the therapy.

Other embodiments provided herein relate to a method of treating a subject with a pharmaceutical composition as described herein. In some embodiments, the subject is refractory to standard diuretic therapy.

Certain subjects who suffer from a cardiac condition, such as congestive heart failure, later develop renal impairment. The present inventors have discovered that if a subject presented with a cardiac condition, and little to no renal impairment, is treated with a pharmaceutical composition as described herein, the onset of renal impairment is delayed or arrested, compared to a subject who receives standard treatment. Thus, some embodiments relate to a method of preventing the deterioration of renal function, delaying the onset of renal impairment, or arresting the progress of renal impairment in a subject comprising identifying a subject in need thereof, and administering a therapeutically effective amount of a KW-3902 derivative of Formula (I), Formula (II), Formula (III), Formula (IV), Formula (V), Formula (VI) or a pharmaceutically acceptable salt, ester, amide, prodrug or metabolite thereof and a non-adenosine modifying diuretic.

The term “treating” or “treatment” does not necessarily mean total cure. Any alleviation of any undesired signs or symptoms of the disease to any extent or the slowing down of the progress of the disease can be considered treatment. Furthermore, treatment may include acts that may worsen the subject's overall feeling of well being or appearance. Treatment may also include lengthening the life of the subject, even if the symptoms are not alleviated, the disease conditions are not ameliorated, or the subject's overall feeling of well being is not improved. Thus, in the context of the present invention, increasing the urine output volume, decreasing the level of serum creatinine, or increasing creatinine clearance, may be considered treatment, even if the subject is not cured or does not generally feel better.

Other embodiments relate to a method of treating a subject suffering from CHF with impaired creatinine clearance, comprising identifying a subject in need thereof, and administering to said subject a therapeutically effective amount of amount of a KW-3902 derivative of Formula (I), Formula (II), Formula (III), Formula (IV), Formula (V), Formula (VI) or a pharmaceutically acceptable salt, ester, amide, prodrug or metabolite thereof and a non-adenosine modifying diuretic.

Still other embodiments relate to a method of improving overall health outcomes, decreasing morbidity rates, or decreasing mortality rates in subjects comprising identifying a subject in need thereof, and administering to said subject a therapeutically effective amount of amount of a KW-3902 derivative of Formula (I), Formula (II), Formula (III), Formula (IV), Formula (V), Formula (VI) or a pharmaceutically acceptable salt, ester, amide, prodrug or metabolite thereof and a non-adenosine modifying diuretic.

Overall health outcomes are determined by various means in the art. For example, improvements in morbidity and/or mortality rates, improvements in the subject's general feelings, improvements in the quality of life, improvements in the level of comfort at the end of life, and the like, are considered when overall health outcome are determined. Mortality rate is the number of subjects who die while undergoing a particular treatment for a period of time compared to the overall number of subjects undergoing the same or similar treatment over the same period of time. Morbidity rates are determined using various criteria, such as the frequency of hospital stays, the length of hospital stays, the frequency of visits to the doctor's office, the dosage of the medication being administered, and the like.

Other embodiments relate to the prevention of the deterioration of renal function in individuals comprising administering a therapeutically effective amount of a KW-3902 derivative of Formula (I), Formula (II), Formula (III), Formula (IV), Formula (V), Formula (VI) or a pharmaceutically acceptable salt, ester, amide, prodrug or metabolite thereof. In some embodiments, the method also includes that administration of a non-adenosine modifying diuretic.

In some embodiments, the subject whose overall health outcome, morbidity and/or mortality rate is being improved suffers from CHF. In other embodiments, the subject suffers from renal impairment. In some embodiments, the subject suffers from CHF and impaired renal function, and/or impaired creatinine clearance.

In embodiments wherein the KW-3902 derivative of Formula (I), Formula (II), Formula (III), Formula (IV), Formula (V), Formula (VI) or a pharmaceutically acceptable salt, ester, amide, prodrug or metabolite thereof, is administered in combination with a non adenosine-modifying diuretic, the administering step comprises administering said KW-3902 derivative and said non adenosine-modifying diuretic nearly simultaneously. These embodiments include those in which the KW-3902 derivative and the non adenosine-modifying diuretic are in the same administrable composition, i.e., a single tablet, pill, or capsule, or a single solution for intravenous injection, or a single drinkable solution, or a single dragee formulation or patch, contains both compounds. The embodiments also include those in which each compound is in a separate administrable composition, but the subject is directed to take the separate compositions nearly simultaneously, i.e., one pill is taken right after the other or that one injection of one compound is made right after the injection of another compound, etc.

In other embodiments the administering step comprises administering the non adenosine-modifying diuretic first and then administering the KW-3902 derivative of Formula (I), (II), (III), (IV), (V), or (V). In yet other embodiments, the administering step comprises administering the KW-3902 derivative of Formula (I), (II), (III), (IV), (V), or (V), first, and then administering the non adenosine-modifying diuretic. In these embodiments, the subject may be administered a composition comprising one of the compounds and then at some time, a few minutes or a few hours, later be administered another composition comprising the other one of the compounds. Also included in these embodiments are those in which the subject is administered a composition comprising one of the compounds on a routine or continuous basis while receiving a composition comprising the other compound occasionally.

The methods disclosed herein are intended to provide treatment for cardiovascular disease, which may include congestive heart failure, hypertension, asymptomatic left ventricular dysfunction, coronary artery disease, or acute myocardial infarction. In some instances, subjects suffering from a cardiovascular disease are in need of after-load reduction. The methods of the present invention are suitable to provide treatment for these subjects as well. Certain subjects who suffer from a cardiac condition, such as congestive heart failure, later develop renal impairment, and/or exhibit impaired creatinine clearance or elevated serum creatinine levels.

Other embodiments relate to the treatment of cardiovascular diseases using a combination of a beta-blocker, and a therapeutically effective amount of a KW-3902 derivative of Formula (I), (II), (III), (IV), (V), or (VI). The present inventors have discovered that the combination of KW-3902 derivatives of Formula (I), (II), (III), (IV), (V), or (VI) and beta blockers is beneficial in either congestive heart failure (CHF) or hypertension, or any of the other indications set forth herein. See, co-pending U.S. application Ser. No. 10/785,446 entitled “Method of Treatment of Disease Using and Adenosine A1 Receptor Antagonist,” filed Feb. 23, 2004, herein expressly incorporated by reference in its entirety.

Beta-blockers are known to have antihypertensive effects. While the exact mechanism of their action is unknown, possible mechanisms, such as reduction in cardiac output, reduction in plasma renin activity, and a central nervous system sympatholytic action, have been put forward. From various clinical studies, it is clear that administration of beta-blockers to subjects with hypertension results initially in a decrease in cardiac output, little immediate change in blood pressure, and an increase in calculated peripheral resistance. With continued administration, blood pressure decreases within a few days, cardiac output remains reduced, and peripheral resistance falls toward pretreatment levels. Plasma renin activity is also reduced markedly in subjects with hypertension, which will have an inhibitory action on the renin-angiotensin system, thus decreasing the after-load and allowing for more efficient forward function of the heart. The use of these compounds has been shown to increase survival rates among subjects suffering from CHF or hypertension. The compounds are now part of the standard of care for CHF and hypertension. The combination of a KW-3902 derivative of Formula (I), (II), (III), (IV), (V), or (VI) and a beta can acts synergistically to further improve the condition of subjects with hypertension or CHF. The diuretic effect of KW-3902 derivatives of Formula (I), (II), (III), (IV), (V), or (VI) especially in salt-sensitive hypertensive subjects along with the blockage of beta adrenergic receptors can decrease blood pressure through two different mechanisms, whose effects build on one another. In addition, most CHF subjects are also on additional diuretics. The combination allows for greater efficacy of other more distally acting diuretics by improving renal blood flow and renal function.

Beta-blockers are well established in the treatment of hypertension. The addition of a KW-3902 derivatives of Formula (I), (II), (III), (IV), (V), or (VI) will further treat hypertension via their diuretic effect from inhibiting sodium reabsorption through the proximal tubule. In addition, since many hypertensive subjects are sodium sensitive, the addition of a KW-3902 derivative of Formula (I), (II), (III), (IV), (V), or (VI) to a beta-blocker will result in further blood pressure reduction. AA₁RA action (e.g., KW-3902 derivatives of Formula (I), (II), (III), (IV), (V), or (VI)) on tubuloglomerular feedback further can improve renal function to result in greater diuresis and lower blood pressure.

In some embodiments concerning methods involving the administration of a KW-3902 derivative of Formula (I), (II), (III), (IV), (V), or (VI) and a beta blocker, the administering step comprises administering said beta-blocker, said AA₁RA, and said anticonvulsant agent nearly simultaneously. These embodiments include those in which the KW-3902 derivative of Formula (I), (II), (III), (IV), (V), or (VI) and the beta-blocker are in the same administrable composition, i.e., a single tablet, pill, or capsule, or a single solution for intravenous injection, or a single drinkable solution, or a single dragee formulation or patch, contains both compounds. The embodiments also include those in which each compound is in a separate administrable composition, but the subject is directed to take the separate compositions nearly simultaneously, i.e., one pill is taken right after the other or that one injection of one compound is made right after the injection of another compound, etc.

In other embodiments the administering step comprises administering one of the beta-blocker and the KW-3902 derivative of Formula (I), (II), (III), (IV), (V), or (VI) first and then administering the other one of the beta-blocker and the KW-3902 derivative of Formula (I), (II), (III), (IV), (V), or (VI). In these embodiments, the subject may be administered a composition comprising one or more of the compounds and then at some time, a few minutes or a few hours, later be administered another composition comprising the other one or more of the remaining compounds. Also included in these embodiments are those in which the subject is administered a composition comprising one of the compounds on a routine or continuous basis while receiving a composition comprising the other compound occasionally.

In another aspect, the invention relates to the treatment of renal and/or cardiac diseases using a combination of a KW-3902 derivative of Formula (I), (II), (III), (IV), (V), or (VI) and an angiotensin converting enzyme (ACE) inhibitor or an angiotensin II receptor blocker (ARB). AA₁RAs (e.g, KW-3902), ACE inhibitors and ARBs have individually been shown to be somewhat effective in the treatment of cardiac disease, such as congestive heart failure, hypertension, asymptomatic left ventricular dysfunction, or acute myocardial infarction, or renal disease, such as diabetic nephropathy, contrast-mediated nephropathy, toxin-induced renal injury, or oxygen free-radical mediated nephropathy.

The present inventors have discovered that the combination of KW-3902 derivatives of Formula (I), (II), (III), (IV), (V), or (VI) and ACE inhibitors or ARBs is beneficial in either congestive heart failure (CHF) or hypertension. See co-pending U.S. application Ser. No. 10/785,446. The use of ACE inhibitors and ARBs in CHF relies on inhibition of renin-angiotensin system. These compounds decrease the after-load, thereby allowing for more efficient forward function of the heart. In addition, renal function is “normalized” or improved such that subjects remove excess fluid more effectively. The use of these compounds has been shown to increase survival rates among subjects suffering from CHF or hypertension. The compounds are now part of the standard of care for CHF and hypertension.

The combination of KW-3902 derivatives of Formula (I), (II), (III), (IV), (V), or (VI) and ACE inhibitors or ARBs acts synergistically to further improve renal function for continued diuresis. In addition, most CHF subjects are also on additional diuretics. The combination allows for greater efficacy of other more distally acting diuretics by improving renal blood flow and renal function.

Both ACE inhibitors and ARBs are well established in the treatment of hypertension via their action through the renin-angiotensin system. The addition of KW-3902 derivatives of Formula (I), (II), (III), (IV), (V), or (VI) will further treat hypertension via its diuretic effect from inhibiting sodium reabsorption through the proximal tubule. In addition, since many hypertensive subjects are sodium sensitive, the addition of a KW-3902 derivative of Formula (I), (II), (III), (IV), (V), or (VI) to an ACE inhibitor or an ARB will result in further blood pressure reduction. AA₁RA action (e.g., KW-3902 derivatives of Formula (I), (II), (III), (IV), (V), or (VI)) on tubuloglomerular feedback further improves renal function to result in greater diuresis and lower blood pressure.

ACE inhibitors and ARBs are also known to prevent some of the renal damage induced by the immunosuppresant, cyclosporin A. However, there is a renal damaging effect despite their use. The present inventors have discovered that the combination ACE inhibitors and ARBs with KW-3902 derivatives of Formula (I), (II), (III), (IV), (V), or (VI) would be more effective in preventing drug-induced nephrotoxicity, such as that induced by cyclosporin A, contrast medium (iodinated), and aminoglycoside antibiotics. In this setting there is renal vasoconstriction that can be minimized by both compounds. In addition, direct negative effects on the tubular epithelium by cyclosporin is less prominent in the setting of adenosine A₁ receptor antagonism, in that blocking A₁ receptors decreases active processes. Furthermore, there are fewer oxidative by-products that are injurious to the tubular epithelium. In addition, the inhibitory effect of AA₁RA (e.g., KW-3902 derivatives of Formula (I), (II), (III), (IV), (V), or (VI)) blockade on the tubuloglomerular feedback mechanism helps preserve function in the setting of nephrotoxic drugs.

It is known that ACE inhibitors and ARBs are beneficial in preventing the worsening of renal dysfunction in diabetics as measured by albuminuria (proteinuria). Once diabetes begins, glucosuria develops and the kidneys begin to actively reabsorb glucose, especially through the proximal convoluted tubule. This active process may result in oxidative stress and begin the disease process of diabetic nephropathy. Early manifestations of this process are hypertrophy and hyperplasia of the kidney. Ultimately, the kidney begins to manifest other signs such as microalbuminuria and decreased function. It is postulated that the active reabsorption of glucose is mediated in part by adenosine A₁ receptors. Blockade of this process by an AA₁RA limits or prevents the early damage manifested in diabetics.

The combination of KW-3902 derivatives of Formula (I), (II), (III), (IV), (V), or (VI) and ACE inhibitors or ARBs, as disclosed herein, works to limit both early and subsequent damage to the kidneys in diabetes. The presently disclosed combinations are given at the time of diagnosis of diabetes or as soon as glycosuria is detected in at risk subjects (metabolic syndrome). The long-term treatment using the combinations of the present invention includes daily administration of the pharmaceutical compositions described herein.

In another aspect, the invention relates to a method of treating cardiovascular disease or renal disease comprising identifying a subject in need of such treatment, and administering a combination of a KW-3902 derivative of Formula (I), (II), (III), (IV), (V), or (VI) and an angiotensin converting enzyme (ACE) inhibitor or an angiotensin II receptor blocker (ARB) to said subject. In certain embodiments, the subject may be a mammal. The mammal may be selected from the group consisting of mice, rats, rabbits, guinea pigs, dogs, cats, sheep, goats, cows, primates, such as monkeys, chimpanzees, and apes, and humans. In some embodiments, the subject is a human.

In some embodiments, the administering step comprises administering said ACE inhibitor or said ARB and said KW-3902 derivative of Formula (I), (II), (III), (IV), (VI), or (V) nearly simultaneously. These embodiments include those in which the KW-3902 derivative of Formula (I), (II), (III), (IV), (V), or (VI) and the ACE inhibitor or ARB are in the same administrable composition, i.e., a single tablet, pill, or capsule, or a single solution for intravenous injection, or a single drinkable solution, or a single dragee formulation or patch, contains both compounds. The embodiments also include those in which each compound is in a separate administrable composition, but the subject is directed to take the separate compositions nearly simultaneously, i.e., one pill is taken right after the other or that one injection of one compound is made right after the injection of another compound, etc.

In other embodiments the administering step comprises administering one of the ACE inhibitor or ARB and the KW-3902 derivative of Formula (I), (II), (III), (IV), (V), or (VI) first and then administering the other one of the ACE inhibitor or ARB and the KW-3902 derivative of Formula (I), (II), (III), (IV), (V), or (VI). In these embodiments, the subject may be administered a composition comprising one of the compounds and then at some time, a few minutes or a few hours, later be administered another composition comprising the other one of the compounds. Also included in these embodiments are those in which the subject is administered a composition comprising one of the compounds on a routine or continuous basis while receiving a composition comprising the other compound occasionally.

The methods of the present invention are intended to provide treatment for cardiovascular disease, which may include congestive heart failure, hypertension, asymptomatic left ventricular dysfunction, or acute myocardial infarction. In some instances, subjects suffering from a cardiovascular disease are in need of after-load reduction. The methods of the present invention are suitable to provide treatment for these subjects as well.

The methods of the present invention are also intended to provide treatment for renal disease, which may include renal hypertrophy, renal hyperplasia, microproteinuria, proteinuria, diabetic nephropathy, contrast-mediated nephropathy, toxin-induced renal injury, or oxygen free-radical mediated nephropathyhypertensive nephropathy, diabetic nephropathy, contrast-mediated nephropathy, toxin-induced renal injury, or oxygen free-radical mediated nephropathy.

Still other aspects relate to methods of treating alkosis using a KW-3902 derivative of Formula (I), (II), (III), (IV), (V), or (VI). Alkalosis is an acid-base disturbance caused by an elevation in plasma bicarbonate (HCO₃⁻) concentration. It is a primary pathophysiologic event characterized by the gain of bicarbonate or the loss of nonvolatile acid from extracellular fluid. The kidney preserves normal acid-base balance by two mechanisms: bicarbonate reclamation, mainly in the proximal tubule, and bicarbonate generation, predominantly in the distal nephron. Bicarbonate reclamation is mediated mainly by a Na⁺—H⁺ antiporter and to a smaller extent by the H-ATPase (adenosine triphosphatase). The principal factors affecting HCO₃⁻ reabsorption include effective arterial blood volume, glomerular filtration rate, potassium, and partial pressure of carbon dioxide. Bicarbonate regeneration is primarily affected by distal Na⁺ delivery and reabsorption, aldosterone, systemic pH, ammonium excretion, and excretion of titratable acid.

There are a number of different types of alkalosis, for instance metabolic alkalosis and respiratory alkalosis. Respiratory alkalosis is a condition that affects mountain climbers in high altitude situations.

To generate metabolic alkalosis, either a gain of base or a loss of acid must occur. The loss of acid may be via the upper gastrointestinal tract or via the kidney. Excess base may be gained by oral or parenteral HCO₃⁻ administration or by lactate, acetate, or citrate administration.

Factors that help maintain metabolic alkalosis include decreased glomerular filtration rate, volume contraction, hypokalemia, and aldosterone excess. Clinical states associated with metabolic alkalosis are vomiting, mineralocorticoid excess, the adrenogenital syndrome, licorice ingestion, diuretic administration, and Bartter's and Gitelman's syndromes.

The two types of metabolic alkalosis (i.e., chloride-responsive, chloride-resistant) are classified based upon the amount of chloride in the urine. Chloride-responsive metabolic alkalosis involves urine chloride levels less than 10 mEq/L, and it is characterized by decreased extracellular fluid (ECF) volume and low serum chloride such as occurs with vomiting. This type responds to administration of chloride salt. Chloride-resistant metabolic alkalosis involves urine chloride levels more than 20 mEq/L, and it is characterized by increased ECF volume. As the name implies, this type resists administration of chloride salt. Ingestion of excessive oral alkali (usually milk plus calcium carbonate) and alkalosis complicating primary hyperaldosteronism are examples of chloride resistant alkalosis.

Many subjects with edematous states are treated with diuretics. Unfortunately, with continued therapy, the subject's bicarbonate level increases and progressive alkalosis may ensue. Diuretics cause metabolic alkalosis by several mechanisms, including (1) acute contraction of the extracellular fluid (ECF) volume (NaCl excretion without HCO₃⁻), thereby increasing the concentration of HCO₃⁻ in the ECF; (2) diuretic-induced potassium and chloride depletion; and (3) secondary aldosteronism. Continued use of the diuretic or either of the latter two factors will maintain the alkalosis.

The addition of an AA₁RA (e.g., a KW-3902 derivative of Formula (I), (II), (III), (IV), (V), or (VI)) allows continued diuresis and maintained renal function without worsening the alkalosis. The AA₁RA inhibits the active resorption of HCO₃⁻ across the proximal tubule of the kidney.

Thus, in one aspect, some embodiments provide methods of treating metabolic alkalosis while reducing the potential of related adverse events occurring, such as seizures or convulsions, comprising identifying a subject in need thereof and administering a KW-3902 derivative of Formula (I), (II), (III), (IV), (V), or (VI) to said subject. In certain embodiments, the subject is suffering from high altitude mountain sickness. In some embodiments, the subject has edema. In some of these embodiments, the subject may be on diuretic therapy. The diuretic may be a loop diuretic, proximal diuretic, or distal diuretic. In other embodiments, the subject suffers from acid loss through the subject's upper gastrointestinal tract, for example, through excessive vomiting. In still other embodiments the subject has ingested excessive oral alkali.

Yet another aspect relates to the treatment of diabetic neuropathy with a KW-3902 derivative of Formula (I), (II), (III), (IV), (V), or (VI). Uncontrolled diabetes causes damage to many tissues of the body. Kidney damage caused by diabetes most often involves thickening and hardening (sclerosis) of the internal kidney structures, particularly the glomerulus (kidney membrane). Kimmelstiel-Wilson disease is the unique microscopic characteristic of diabetic nephropathy in which sclerosis of the glomeruli is accompanied by nodular deposits of hyaline.

The glomeruli are the site where blood is filtered and urine is formed. They act as a selective membrane, allowing some substances to be excreted in the urine and other substances to remain in the body. As diabetic nephropathy progresses, increasing numbers of glomeruli are destroyed, resulting in impaired kidney functioning. Filtration slows and protein, namely albumin, which is normally retained in the body, may leak in the urine. Albumin may appear in the urine for 5 to 10 years before other symptoms develop. Hypertension often accompanies diabetic nephropathy.

Diabetic nephropathy may eventually lead to the nephrotic syndrome (a group of symptoms characterized by excessive loss of protein in the urine) and chronic renal failure. The disorder continues to progress, with end-stage renal disease developing, usually within 2 to 6 years after the appearance of renal insufficiency with proteinuria.

The mechanism that causes diabetic nephropathy is unknown. It may be caused by inappropriate incorporation of glucose molecules into the structures of the basement membrane and the tissues of the glomerulus. Hyperfiltration (increased urine production) associated with high blood sugar levels may be an additional mechanism of disease development.

Diabetic nephropathy is the most common cause of chronic renal failure and end stage renal disease in the United States. About 40% of people with insulin-dependent diabetes will eventually develop end-stage renal disease. 80% of people with diabetic nephropathy as a result of insulin-dependent diabetes mellitus (IDDM) have had this diabetes for 18 or more years. At least 20% of people with non-insulin-dependent diabetes mellitus (NIDDM) will develop diabetic nephropathy, but the time course of development of the disorder is much more variable than in IDDM. The risk is related to the control of the blood-glucose levels. Risk is higher if glucose is poorly controlled than if the glucose level is well controlled.

Diabetic nephropathy is generally accompanied by other diabetic complications including hypertension, retinopathy, and vascular (blood vessel) changes, although these may not be obvious during the early stages of nephropathy. Nephropathy may be present for many years before nephrotic syndrome or chronic renal failure develops. Nephropathy is often diagnosed when routine urinalysis shows protein in the urine.

Current treatments for diabetic nephropathy include administration of angiotensin converting enzyme inhibitors (ACE Inhibitors) during the more advanced stages of the disease. Currently there is no treatment in the earlier stages of the disease since ACE inhibitors may not be effective when the disease is symptom-free (i.e., when the subject only shows proteinuria).

Although the mechanism implicated in early renal disease in diabetics is that of hyperglycemia, a potential mechanism may be related to the active reabsorption of glucose in the proximal tubule. This reabsorption is dependent in part on adenosine A₁ receptors.

AA₁RAs act on the afferent arteriole of the kidney to produce vasodilation and thereby improve renal blood flow in subjects with diabetes. This ultimately allows for increased GFR and improved renal function. In addition, AA₁RAs inhibit the reabsorption of glucose in the proximal tubule in subjects with newly diagnosed diabetic mellitus or in subjects at risk for the condition (metabolic syndrome).

Thus, in one aspect, the present invention relates to a method of treating diabetic nephropathy comprising identifying a subject in need thereof and administering a KW-3902 derivative of Formula (I), (II), (III), (IV), (V), or (VI) to said subject. In certain embodiments the subject is pre-diabetic, whereas in other embodiments the subject is in early stage diabetes. In some embodiments the subject suffers from insulin-dependent diabetes mellitus (IDDM), whereas in other embodiments the subject suffers from non-insulin-dependent diabetes mellitus (NIDDM).

In certain embodiments, the methods of the present invention are used to prevent or reverse renal hypertrophy. In other embodiments, the methods of the present invention are used to prevent or reverse renal hyperplasia. In still other embodiments, the methods of the present invention are used to ameliorate microproteinuria or proteinuria.

Before people develop type II diabetes, i.e., NIDDM, they almost always have “pre-diabetes.” Pre-diabetic subjects have blood glucose levels that are higher than normal but not yet high enough to be diagnosed as diabetes. For instance, the blood glucose level of pre-diabetic subjects is between 110-126 mg/dL, using the fasting plasma glucose test (FPG), or between 140-200 mg/dL using the oral glucose tolerance test (OGTT). Blood glucose levels below 110 or 140, using FPG or OGTT, respectively, is considered normal, whereas individuals with blood glucose levels higher than 126 or 200, using FPG or OGTT, respectively, are considered diabetic. The methods of the present invention can be practiced with any compound that antagonizes adenosine A₁ receptors.

In certain aspects, the methods of the present invention can be practiced using a combination therapy, i.e., where the KW-3902 derivative of Formula (I), (II), (III), (IV), (V), or (VI) is administered to the subject in combination with a second compound. In certain embodiments the second compound may be selected from a protein kinase C inhibitor, an inhibitor of tissue proliferation, an antioxidant, an inhibitor of glycosylation, and an endothelin B receptor inhibitor.

Having now generally described the invention, the same will become better understood by reference to certain specific examples which are included herein for purposes of illustration only and are not intended to be limiting unless other wise specified. All referenced publications and patents are incorporated, in their entirety by reference herein.

### EXAMPLES

### Example 1

### Treatment of Individuals with Fluid Overload and Renal Impairment

A double-blind, randomized multi-center, placebo controlled study is conducted as follows: Males and females at least 18 years of age with New York Heart Association Class II-IV CHF and having an estimated creatinine clearance between 20 mL/min and 80 mL/min are identified. The subjects are taking an oral loop diuretic.

Study visits include pre-treatment days −2 to −1, days 1 to 3 of the Treatment Period, day 4/early Termination and a follow up contact at day 30. Procedures and observations include medical history, physical examination, classification of CHF, vital signs, body weight, CHF signs and symptom scores, Holter monitor recording, chest X-ray, CBC chemistries, creatinine clearance, fluid intake, and urine output.

On treatment days, individuals receive a KW-3902 derivative of Formula (I), (II), (III), (IV), (V), or (VI) intravenously over 120 minutes at a dose between about 2.5 mg to about 100 mg vs. placebo as both monotherapy and concomitant therapy with diuretics. KW-3902 derivatives of Formula (I), (II), (III), (IV), (V), or (VI) (or placebo) are administered on days 1 through 3. On day 1, the KW-3902 derivatives of Formula (I), (II), (III), (IV), (V), or (VI) (or placebo) is administered as a monotherapy. 6 hours after administration of the KW-3902 derivative, IV loop diuretic is given to all treatment groups as needed. On days 2 and 3 KW-3902 derivatives are administered as combination therapy with intravenous furosemide, if clinically indicated. Final laboratory data are collected on day 4 or early termination. Follow-up phone contact is conducted on day 30.

Individuals receiving as low as 2.5 mg KW-3902 derivatives of Formula (I), (II), (III), (IV), (V), or (VI) exhibit an improvement in kidney function as measured by serum creatinine levels compared to the baseline levels. This effect is grater than the effect seen in individuals who receive placebo.

The combination of KW-3902 derivatives of Formula (I), (II), (III), (IV), (V), or (VI) and non adenosine-modifying diuretics such as furosemide has a synergistic beneficial effect on diuresis, as measured by urine output. Individuals receiving KW-3902 derivatives of Formula (I), (II), (III), (IV), (V), or (VI) also require less non adenosine-modifying diuretic

### Example 2

### Treatment of Individuals with Fluid Overload and Renal Impairment

A patient with fluid overload, as manifested by peripheral edema, dyspnea, and/or other signs or symptoms presents to the hospital, clinic, or doctor's office. The patient also shows some degree of renal impairment. In addition to standard of care therapy which would include IV diuretics, e.g., IV furosemide, bumetanide and/or oral metolazone, the patient is also given a does of a KW-3902 derivative of Formula (I), (II), (III), (IV), (V), or (VI) of between about 2.5 mg to about 100 mg (e.g., 10 mg, 20 mg, 30 mg, 40 mg, 50 mg, 60 mg, 70 mg, 80 mg, 90 mg, or 100 mg or more) in injectable form. The patient is administered 30 mg of a KW-3902 derivatives\ of Formula (I), (II), (III), (IV), (V), or (VI) and 40 mg of furosemide at 24 hour intervals or more frequently as needed. The patient's fluid intake and output, urine volume, serum and urine creatinine levels, electrolytes and cardiac function are monitored.

At the discretion of the attending physician, the dosage of the KW-3902 derivative of Formula (I), (II), (III), (IV), (V), or (VI) can be increased or decreased either during the treatment or as the initial dose. In addition, the dosage of furosemide can be increased to 60 mg, 80 mg, 100 mg, 120 mg, 140 mg, or 160 mg either during the treatment or as the initial dose, or furosemide can be given as a continuous infusion.

### Example 3

### Treatment of Individuals Refractory to Standard IV Diuretic Therapy

A double-blind, randomized, multi-site, placebo controlled study is conducted as follows: Subjects presenting with congestive heart failure having an estimated creatinine clearance between 20 mL/min and 80 mL/min and who are refractory to high dose diuretic therapy are randomized to treatment groups receiving KW-3902 derivatives of Formula (I), (II), (III), (IV), (V), or (VI) or placebo. Doses of 10 mg, 30 mg, and 60 mg KW-3902 derivatives of Formula (I), (II), (III), (IV), (V), or (VI) intravenous or placebo are administered once over 120 minutes. Changes in urine output are measured hourly. The creatinine clearance rate is measured every three hours.

All doses of KW-3902 derivatives result in increased hourly urine volume over the ensuing 9 hours compared to placebo. Subjects administered KW-3902 derivatives of Formula (I), (II), (III), (IV), (V), or (VI) also exhibit an improvement in creatinine clearance.

A hospitalized patient who has been treated with maximum amounts of IV diuretic and is still symptomatic, fluid overloaded, or whose urine output is less than fluid intake is evaluated for further treatment. A 10, 20, 30, 40, 50, 60, 70, 80, 90, or 100 mg dose of a KW-3902 derivative of Formula (I), (II), (III), (IV), (V), or (VI) in injectable form is infused through the IV line. The patient receives continued treatment with furosemide, and also receives 10 mg of KW-3902 at 6 hour intervals, or more or less frequently as needed. The patient's fluid intake and output, urine volume, serum and urine creatinine levels, electrolytes and cardiac function are monitored.

At the discretion of the attending physician, the dosage of the KW-3902 derivative can be increased or decreased either during the treatment or as the initial dose, or furosemide can be given as a continuous infusion.

### Example 4

### Treatment of Individuals Refractory to Standard IV Diuretic Therapy

A hospitalized patient who has been treated with maximum amounts of IV diuretic and is still symptomatic, fluid overloaded, or whose urine output is less than fluid intake is evaluated for further treatment. A 10 mg, 20 mg, 30 mg, 40 mg, 50 mg, 60 mg, 70 mg, 80 mg, 90 mg, or 100 mg dose of a KW-3902 derivative of Formula (I), (II), (III), (IV), (V), or (VI) in injectable form is infused through the IV line. The patient receives continued treatment with furosemide, and also receives doses of KW-3902 derivatives of of Formula (I), (II), (III), (IV), (V), or (VI) at 6 hour intervals, or more or less frequently as needed. The patient's fluid intake and output, urine volute, serum and urine creatinine levels, electrolytes and cardiac function are monitored.

At the discretion of the attending physician, the dosage of KW-3902 derivatives can be increased or decreased either during the treatment or as the initial dose, or furosemide can be given as a continuous infusion.

### Example 5

### Treatment of Individuals with Fluid Overload

A patient with fluid overload, as manifested by peripheral edema, dyspnea, and/or other signs or symptoms presents to the hospital, clinic, or doctor's office. In addition to standard of care therapy which would include IV diuretics, e.g., IV furosemide, bumetanide and/or oral metolazone, the patient is also given 2.5 mg-100 mg (e.g,. 10 mg, 20 mg, 30 mg, 40 mg, 50 mg, 60 mg, 70 mg, 80 mg, 90 mg, or 100 mg or more) of a KW-3902 derivative of Formula (I), (II), (III), (IV), (V), or (VI) in injectable form. The patient is administered a dose of KW-3902 derivative and 40 mg of furosemide at 24 hour intervals, or furosemide can be given as a continuous infusion. The patient's fluid intake and output, urine volume, serum and urine creatinine levels, electrolytes and cardiac function are monitored.

At the discretion of the attending physician, the dosage of the KW-3902 derivative can be increased or decreased either during the treatment or as the initial dose. In addition, the dosage of furosemide can be increased to 60 mg, 80 mg, 100 mg, 120 mg, 140 mg, or 160 mg either during the treatment or as the initial dose. This treatment can be used for patients whether or not they suffer from renal impairment.

### Example 6

### Treatment of Individuals with Fluid Overload and Impaired Renal Function

A patient with fluid overload, as manifested by peripheral edema, dyspnea, and/or other signs or symptoms presents himself to the physician's office or clinic. The patient has been on a therapy regimen that includes oral diuretics and, in addition, to needing a higher dose of diuretics to manage his/her fluid balance, the patient is now showing impaired renal function. The patient is prescribed a dose of 5 mg, 10 mg, 20 mg, 30 mg, 40 mg, 50 mg, 60 mg, 70 mg, 80 mg, 90 mg, or 100 mg or more of a KW-3902 derivative of Formula (I), (II), (III), (IV), (V), or (VI) to be taken orally, once daily, concurrent with other diuretic therapy. The patient's fluid intake and output, urine volume, serum and urine creatinine levels, electrolytes and cardiac function are monitored.

At the discretion of the attending physician, the dosage of the KW-3902 derivative can be increased or decreased either during the treatment or as the initial dose. In addition, the dosage of furosemide can be increased to 60 mg, 80 mg, 100 mg, 120 mg, 140 mg, or 160 mg either during the treatment or as the initial dose.

### Example 7

### Treatment of Individuals with Fluid Overload

A patient with fluid overload, as manifested by peripheral edema, dyspnea, and/or other signs or symptoms presents to the physician's office or clinic. The patient has been on a therapy regimen that includes oral diuretics and needs a higher dose of diuretics to manage his/her fluid balance. To delay or prevent the onset of renal impairment and/or to delay the need to use higher dosages of standard diuretics, the patient is prescribed 5 mg, 10 mg, 20 mg, 30 mg, 40 mg, 50 mg, 60 mg, 70 mg, 80 mg, 90 mg, or 100 mg or more of a KW-3902 derivative of Formula (I), (II), (III), (IV), (V), or (VI) to be taken orally, once daily, concurrent with their diuretic therapy. The patient's fluid intake and output, urine volume, serum and urine creatinine levels, electrolytes and cardiac function are monitored.

At the discretion of the attending physician, the dosage of KW-3902 can be increased or decreased either during the treatment or as the initial dose. In addition, the dosage of furosemide can be increased to 60 mg, 80 mg, 100 mg, 120 mg, 140 mg, or 160 mg either during the treatment or as the initial dose.

### Example 8

### Treatment of Individuals with Congestive Heart Failure

A patient with congestive heart failure presents to the physician's office or clinic. The patient is put on a therapy regimen that includes oral diuretics to manage his/her fluid balance. To delay or prevent the onset of renal impairment and/or to delay the need to use higher dosages of standard diuretics, the patient is also prescribed 5 mg, 10 mg, 20 mg, 30 mg, 40 mg, 50 mg, 60 mg, 70 mg, 80 mg, 90 mg, or 100 mg or more of a KW-3902 derivative of Formula (I), (II), (III), (IV), (V), or (VI) to be taken orally, once daily, concurrent with their diuretic therapy. The patient's fluid levels, urine volume, serum and urine creatinine levels, electrolytes and cardiac function are monitored.

At the discretion of the attending physician, the dosage of the KW-3902 derivative can be increased or decreased either during the treatment or as the initial dose. In addition, the dosage of furosemide can be increased to 60 mg, 80 mg, 100 mg, 120 mg, 140 mg, or 160 mg either during the treatment or as the initial dose.

### Example 9

### Improving Health Outcomes for of Individuals with Congestive Heart Failure

A patient with congestive heart failure presents to the physician's office or clinic. The patient is put on a therapy regimen that includes oral diuretics to manage his/her fluid balance. To improve overall health outcomes (i.e., morbidity or mortality rates due to CHF), the patient is also prescribed 5 mg, 10 mg, 20 mg, 30 mg, 40 mg, 50 mg, 60 mg, 70 mg, 80 mg, 90 mg, or 100 mg or more of a KW-3902 derivative of Formula (I), (II), (III), (IV), (V), or (VI) to be taken orally, once daily, concurrent with their diuretic therapy, or similar doses of KW-3902 derivative is administered to the patient intravenously. The patient's fluid levels, urine volume, serum and urine creatinine levels, electrolytes and cardiac function are monitored.

At the discretion of the attending physician, the dosage of KW-3902 can be increased or decreased either during the treatment or as the initial dose. In addition, the dosage of furosemide can be increased to 60 mg, 80 mg, 100 mg, 120 mg, 140 mg, or 160 mg either during the treatment or as the initial dose.

What is claimed is:

1. A xanthine compound represented by the Formula I, 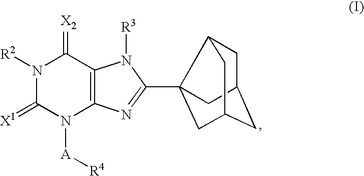
wherein R² represents a hydrogen, lower alkyl, allyl, propargyl, or hydroxyl-substituted, oxo-substituted, or unsubstituted lower alkyl, and
R³ represents hydrogen or lower alkyl,
wherein each of X¹ and X² independently represents oxygen or sulfur,
wherein A is a substituted or unsubstituted substituent selected from the group consisting of a branched or unbranched alkyl, an alkenyl, an alkynyl, an ester, an ether, a thioether, an amide or an aryl amide,
and wherein R⁴ represents an aryl, heteroaryl, heterocyclyl, cycloalkyl, cycloalkenyl, cycloalkynyl, arylalkyl, heteroarylalkyl, and heterocyclylalkyl, wherein each of the foregoing is substituted with a charged or polar moiety,
or a pharmaceutically acceptable salt, ester, amide, metabolite, or prodrug thereof.

2. The compound of claim 1, wherein R⁴ is an aryl group.

3. The compound of claim 1, wherein A is an amide group.

4. The compound of claim 1, wherein the amide group is propyl amide.

5. The compound of claim 1, wherein the charged moiety is in the para position.

6. The compound of claim 1, wherein said polar or charged moiety is selected from the group consisting of SO₃, SO₂H, PO₃, PO₂H, NO₃, NO₂H, CF₃, CH₂F, CHF₂F₂, cyano isocyano, amide, guanadinium, and amino.

7. The compound of claim 2, wherein said polar or charged moiety is SO₃.

8. The compound of claim 3, wherein said polar or charged moiety is SO₃.

9. The compound of claim 6, wherein said polar or charged moiety is SO₃.

10. The compound of claim 1, wherein the compound is

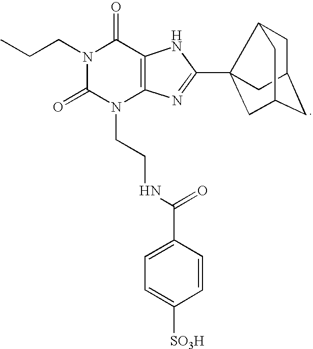

11. A pharmaceutical composition comprising the xanthine compound of claim 1 and a pharmaceutically acceptable carrier.

12. A pharmaceutical composition comprising the xanthine compound of claim 10 and a pharmaceutically acceptable carrier.

13. A xanthine compound represented by the Formula II, 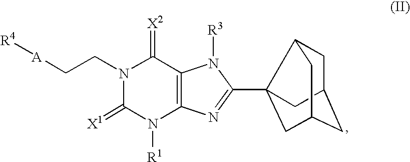
wherein R¹ represents a hydrogen, lower alkyl, allyl, propargyl, or hydroxyl-substituted, oxo-substituted, or unsubstituted lower alkyl, and
R³ represents hydrogen or lower alkyl,
wherein each of X¹ and X² independently represents oxygen or sulfer,
wherein A is a substituted or unsubstituted substituent selected from the group consisting of a branched or unbranched alkyl, an alenyl, an alkynyl, an ester, an ether a thiother, an amide or an aryl amide,
and wherein R⁴ represents an aryl, heteroaryl, heterocyclyl, cycloalkyl, cycloalkenyl, cycloalkynyl, arylalkyl, heteroarylalkyl, and heterocyclylalkyl, wherein each of the foregoing is substituted with a charged or polar moiety,
or a pharmaceutically acceptable salt, ester, amide, metabolite, or prodrug thereof.

14. The compound of claim 13, wherein R⁴ is an aryl group.

15. The compound of claim 13, wherein A is an amide group.

16. The compound of claim 13, wherein the amide group is propyl amide.

17. The compound of claim 13, wherein the charged moiety is in the para postion.

18. The compound of claim 13, wherein said polar or charged moiety is selected from the group consisting of SO₃, SO₂H, PO₃, PO₂H, NO₃, NO₂H, CF₃, CH₂F, CHF₂, cyano, isocyano, amide, guanadinium, and amino.

19. The compound of claim 14, wherein said polar or charged moiety is SO₃.

20. The compound of claim 15, wherein said polar or charged moiety is SO₃.

21. The compound of claim 18, wherein said polar or charged moiety is SO₃.

22. The compound of claim 13, wherein the compound is

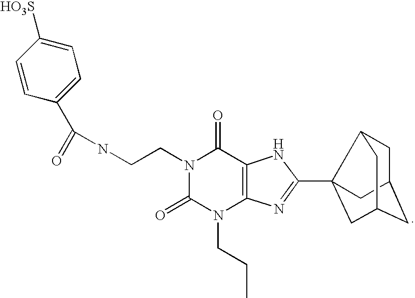

23. A pharmaceutical composition comprising the xanthine compound of claim 13 and a pharmaceutically acceptable carrier.

24. A pharmaceutical composition comprising the xanthine compound of claim 22 and a pharmaceutically acceptable carrier.

25. A xanthine compound represented by the Formula III, 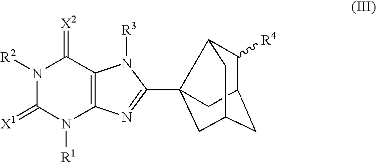
wherein each of R¹, R² independently represents hydrogen, lower alkyl, allyl, propargyl, or hydroxy-substituted, oxo-substituted or unsubstituted lower alkyl, and R³ represents hydrogen or lower alkyl,
and wherein each of X¹ and X² independently represents oxygen or sulfur, and wherein R⁴ represents an aryl, heteroaryl, heterocyclyl, cycloalkyl, cycloalkenyl, cycloalkynyl, arylalkyl, heteroarylalkyl, and heterocyclylalkyl, wherein each of the foregoing is substituted with a charged or polar moiety,
or a pharmaceutically acceptable salt, ester, amide, metabolite, or prodrug thereof.

26. The compound of claim 25, wherein R⁴ is an aryl group.

27. The compound of claim 25, wherein said polar or charged moiety is in the para position.

28. The compound of claim 25, wherein said polar or charged moiety is selected from the group consisting of SO₃, SO₂H, PO₃, PO₂H, NO₃, NO₂H, CF₃, CH₂F, CHF₂, cyano, isocyano, amido, guanadinium, and amino.

29. The compound of claim 29, wherein said polar or charged moiety is SO₃.

30. The compound of claim 27, wherein said polar or charged moiety is SO₃.

31. A pharmaceutical composition comprising the xanthine compound of claim 25 and a pharmaceutically acceptable carrier.

32. The xanthine compound of claim 25, wherein the compound is

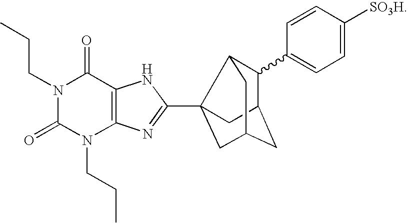

33. A xanthine compound represented by the Formula IV: 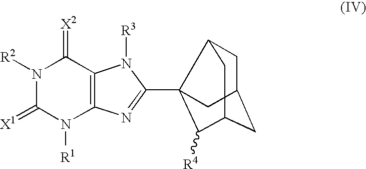
wherein each of R¹, R² independently represents hydrogen, lower alkyl, allyl, propargyl, or hydroxy-substituted, oxo-substituted or unsubstituted lower alkyl, and R³ represents hydrogen or lower alkyl,
and wherein each of X¹ and X² independently represents oxygen or sulfur,
and wherein R⁴ represents an aryl, heteroaryl, heterocyclyl, cycloalkyl, cycloalkenyl, cycloalkynyl, arylalkyl, heteroarylalkyl, and heterocyclylalkyl, wherein each of the foregoing is substituted with a charged or polar moiety,
or a pharmaceutically acceptable salt, ester, amide, metabolite, or prodrug thereof.

34. The compound of claim 33, wherein R⁴ is an aryl group.

35. The compound of claim 33, wherein said polar or charged moiety is in the para position.

36. The compound of claim 33, wherein said polar or charged moiety is selected from the group consisting of SO₃, SO₂H, PO₃, PO₂H, NO₃, NO₂H, CF₃, CH₂F, CHF₂, cyano, isocyano, amido, guanadinium, and amino.

37. The compound of claim 36, wherein said polar or charged moiety is SO₃.

38. The compound of claim 34, wherein said polar or charged moiety is SO₃.

39. A pharmaceutical composition comprising the xanthine compound of claim 33 and a pharmaceutically acceptable carrier.

40. The compound of claim 33, wherein the compound is:

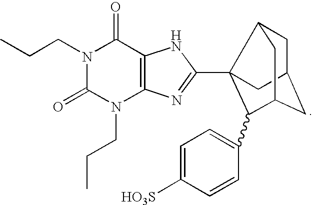

41. A xanthine compound represented by the Formula V: 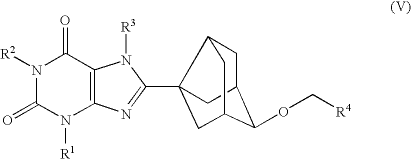
wherein each of R¹, R² independently represents hydrogen, lower alkyl, allyl, propargyl, or hydroxy-substituted, oxo-substituted or unsubstituted lower alkyl, and R³ represents hydrogen or lower alkyl,
and wherein each of X¹ and X² independently represents oxygen or sulfur, and wherein R⁴ represents an aryl, heteroaryl, heterocyclyl, cycloalkyl, cycloalkenyl, cycloalkynyl, arylalkyl, heteroarylalkyl, and heterocyclylalkyl, wherein each of the foregoing is substituted with a charged or polar moiety,
or a pharmaceutically acceptable salt, ester, amide, metabolite, or prodrug thereof.

42. The compound of claim 41, wherein R⁴ is an aryl group.

43. The compound of claim 41, wherein said polar or charged moiety is in the para position.

44. The compound of claim 41, wherein said polar or charged moiety is selected from the group consisting of SO₃, SO₂H, PO₃, PO₂H, NO₃, NO₂H, CF₃, CH₂F, CHF₂, cyano, isocyano, amide, guanadinium, and amino.

45. The compound of claim 44, wherein said polar or charged moiety is SO₃.

46. The compound of claim 42, wherein said polar or charged moiety is SO₃.

47. A pharmaceutical composition comprising the xanthine compound of claim 41 and a pharmaceutically acceptable carrier.

48. The compound of claim 41, wherein the compound is:

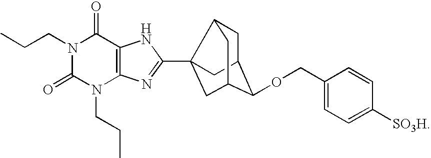

49. A xanthine compound represented by the Formula (VI): 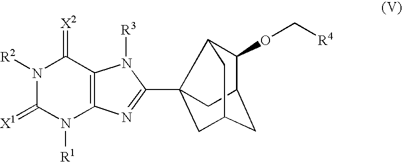
wherein each of R¹, R² independently represents hydrogen, lower alkyl, allyl, propargyl, or hydroxy-substituted, oxo-substituted or unsubstituted lower alkyl, and R³ represents hydrogen or lower alkyl,
and wherein each of X¹ and X² independently represents oxygen or sulfur,
and wherein R⁴ represents an aryl, heteroaryl, heterocyclyl, cycloalkyl, cycloalkenyl, cycloalkynyl, arylalkyl, heteroarylalkyl, and heterocyclylalkyl, wherein each of the foregoing is substituted with a charged or polar moiety,
or a pharmaceutically acceptable salt, ester, amide, metabolite, or prodrug thereof.

50. The compound of claim 49, wherein R⁴ is an aryl group.

51. The compound of claim 49, wherein said polar or charged moiety is in the para position.

52. The compound of claim 49, wherein said polar or charged moiety is selected from the group consisting of SO₃, SO₂H, PO₃, PO₂H, NO₃, NO₂H, CF₃, CH₂F, CHF₂, cyano, isoacyano, amide, guanadinium, and amino.

53. The compound of claim 52, wherein said polar or charged moiety is SO₃.

54. The compound of claim 50, wherein said polar or charged moiety is SO₃.

55. A pharmaceutical composition comprising the xanthine compound of claim 49 and a pharmaceutically acceptable carrier.

56. The compound of claim 49, wherein the compound is: [chemical structure]
